(19) United States
(12) Patent Application Publication
(10) Pub. No.: US 2011/0081320 A1
(43) Pub. Date: Apr. 7, 2011

(54) Treatment/Cure of Autoimmune Disease

(75) Inventors: Fredrick C. Westall, Temecula, CA (US); Richard Blankenbecler, Las Vegas, NV (US); Brian C. Lue, Mountain View, CA (US)

(73) Assignee: NUBIOME, INC., Mountain View, CA (US)

(21) Appl. No.: 12/898,313

(22) Filed: Oct. 5, 2010

Related U.S. Application Data
(60) Provisional application No. 61/278,333, filed on Oct. 6, 2009.

(51) Int. Cl. A61K 38/48; A61P 25/00; A61P 19/02; A61P 21/04; A61P 29/00; A61P 1/04; A61K 35/74; A61P 3/00

(52) U.S. Cl. 424/93.4; 424/94.64; 424/94.67

(57) ABSTRACT

An embodiment entails administering an effective amount of a product such as a protease or a source of a protease that destroys or deactivates an immunogen, mimic or antigen specific to a particular autoimmune disease before it encounters the immune system of a patient.

This patent application relates to U.S. Provisional Application No. 61/278,333 filed Oct. 6, 2009 from which priority is claimed under 35 USC §119(e), and which provisional application is incorporated herein in its entirety.

### TECHNICAL FIELD

One or more embodiments of the present invention provide methods for treating or curing autoimmune diseases.

### BACKGROUND

Chronic autoimmune diseases include Multiple Sclerosis, Ulcerative Colitis, Lupus Erythematosus, Myasthenia Gravis, Uveoretinitis, Arthritis, Autoimmune Diabetes, and Autoimmune Neuritis, for example, Guillain Barre Syndrome. Many of these diseases are characterized by unpredictable clinical exacerbations and remissions.

These diseases may be initiated by microorganisms which contain an amino acid sequence within one of its proteins that mimics structurally a similar sequence within a protein in human tissue (for example, see an article entitled “Sequence Homology Between Certain Viral Proteins and Proteins Related to Encephalomyelitis and Neuritis” by U. Jahnke, F. D. Fisher and E. C. Alvord, Jr. in Science, 229 pp. 282-284 (1985) and an article entitled “Amino Acid Homology Between the Encephalitogenic Site of MBP and Virus: Mechanism for Autoimmunity” by R. S. Fujinami and M. B. Oldstone in Science, 230 pp. 1043-1045 (1985)). When such a microorganism invades an individual, it activates the immune system against the mimic. The activated immune system then recognizes the similar sequence within the protein in the human tissue, and initiates an immune attack. This immune attack on its own tissue results in the autoimmune disease.

A. Immunogens Producing Autoimmune Diseases: The identities of immunogens, antigens, or mimics that trigger autoimmune diseases are unknown. However, each human autoimmune disease has an animal autoimmune disease counterpart. Further, a protein or proteins from animal tissue that appear to be capable of activating these animal autoimmune diseases have been identified. To test whether an agent is an immunogen for the animal autoimmune disease, the suspected agent is combined with an immunological adjuvant and injected into a test animal. If clinical signs develop in the animal which are similar to those for the human disease, the protein is considered to be a potential immunogen responsible for the human disease. In addition, regions, i.e., sequences, within these proteins have been identified which themselves have been shown to trigger the autoimmune response for the disease in animals. Further, the contributions to induction of the autoimmune response of the individual amino acids within these sequences have also been determined. By knowing the contribution to disease induction of each amino acid within an immunogen, one can identify potential mimics.

B. Mimics: To identify a potential autoimmune mimic, one first must have a defined immunogen. It is not sufficient that the mimic have some or most of the same amino acids in its sequence. Since each amino acid makes a different contribution towards the autoimmune process, the mimic's sequence must match these contributions. Table 1 in Appendix I lists a series of chemically defined peptide immunogens which are able to initiate a variety of experimental autoimmune diseases such as, for example, experimental allergic encephalomyelitis (EAE)—a model for multiple sclerosis; experimental allergic uveoretinitis (EAU)—a model for a variety human retinitis maladies; experimental myasthenia gravis (EMG)—a model for myasthenia gravis; and experimental systemic lupus erythematosus (ELSE)—a model for lupus.

The immunogens responsible for experimental lupus contain a high concentration of proline, e.g., PPPGMRPP. This, like the other immunogens, should be particularly difficult to hydrolyze. Furthermore it has been reported that in both the experimental lupus and the corresponding human lupus, proteases involved in proline peptide hydrolysis are depressed (for example, see articles: “Abnormality of the post-proline cleaving enzyme activity in mice with systemic lupus erythematosus-like syndrome” by T. Aoyagi et al. in J. Appl. Biochem., 7 pp. 273-81 (1985); “Decreased activities of serine proteinases in spleen of various murine models of systemic lupus erythematodes” by T. Aoyagi, T. Wada, H. P. Daskalov, F. Kojima, et al. in Biotechnol. Appl. Biochem., 9 pp. 362-7 (1987); “Dissociation between serine proteinases and proline related enzymes in spleen of MRL mouse as a model of systemic lupus erythematodes” by T. Aoyagi, T. Wada, H. P. Daskalov, F. Kojima, et al. in Biochem. Int. 14 pp. 435-41 (1987); and “Activities of Dipeptidyl Peptidase II and Dipeptidyl Peptidase IV in Mice and Lupus Erythematosus-Like Syndrome and in Patients with Lupus Erythematosus and Rheumatoid Arthritis” by M. Hagihara, M. Ohhashi and T. Nagatsu in Clin. Chem., 33 pp 1463-1465 (1987).

Several other disease such as, ulcerative colitis, anorexia, celiac disease, rheumatoid arthritis and even autism, are appear also to have autoimmune aspects.

It is possible that no single etiological factor or agent is responsible for the onset and perpetuation of ulcerative colitis. However, some believe that ulcerative colitis is initiated by an autoimmune response against some component of the intestinal mucosa. An article by Das et al. (see an article entitled “Autoimmunity to cytosketal protein tropomyosin. A clue to the pathogenic mechanism for ulcerative colitis” by K. M. Das, A. Dasgupta, A. Mandal, and X. Geng in J. Immunol., 150 pp. 2487-93 (1993)) has suggested that tropomyosin 3 isoform V, a 247 amino acid protein, is such a component. An article by Mirza et al. (see an article entitled “Autoimmunity Against Human Tropomyosin Isoforms in Ulcerative Colitis” by Z. K. Mirza, S. Bhagyalakshmi, J. J.-C. Lin, P. S. Amenta and K. M. Das in Inflamm. Bowel Dis., 12 pp. 1036-1043 (2006)) has shown that tropomyosin 3 isoform V is the predominant isoform in the epithelium of colon and extra-intestinal organs commonly involved in ulcerative colitis. An article by Kesari et al. (see an article entitled “Externalization of Tropomyosin Isoform 5 in Colon Epithelial Cells” by K. Y. Kesari, N. Yoshizaki, X. Geng. J. J.-C. Lin and K. M. Das in Clin. Exp. Immunol., 118 pp. 219-227 (1999)) has shown that tropomyosin 3 isoform V not only is located intracellularly within colon epithelial cells, but more important, is normally released from these cells into the colon. Released tropomyosin 3 isoform V can interact with mimic activated gut immune cells, and the antibody associated with tropomyosin blocks its activity in vitro. An article by Das et al. (see an article entitled “Autoimmunity to Cytosketal Protein Tropomyosin. A Clue to the Pathogenic Mechanism for Ulcerative Colitis” by K. M. Das, A. Dasgupta, A. Mandal and X. Geng in J. Immunol., 150 pp. 2487-93 (1993)) has suggested that tropomyosin 3 isoform V plays an integral part in the pathology of ulcerative colitis. An article by Geng et al. (see an article entitled “Tropomyosin Isoforms in Intestinal Mucosa: Production of Autoantibodies to Tropomyosin Isoforms in Ulcerative Colitis” by X. Geng, L. Biancone, H. H. Dai, J. J. Lin, N. Yoshizaki, A. Dasgupta, F. Pallone and K. M. Das in Gastrolenterol., 114 pp. 912-22 (1998)) has found antibodies directed against this protein in ulcerative colitis in both the mucosa and sera. If its presence were used as a diagnostic test to distinguish ulcerative colitis from Crohn's disease, the test would have 100% specificity (see, for example, an article entitled “Antibody to Tropomyosin Isoform 5 and Complement Induce the Lysis of Coloncytes in Ulcerative Colitis” by E. C. Ebert, X. Geng, M. Baipai, Z. Pan, E. Tater and K. M. Das in Am. J. Gastroenterol., 104 pp. 2996-3003 (2009)). Autoantibodies against human tropomyosin 3 isoform V in ulcerative colitis (see, for example, an article entitled “Autoantibodies Against Human Tropomyosin Isoform 5 in Ulcerative Colitis Destroys Colonic Epithelial Cells Through Antibody and Compliment-Mediated Lysis” by E. C. Ebert, X. Geng, J. Lin and K. M. Das in Cell. Immunol., 244 pp. 43-9 (2006)) destroy colonic epithelial cells through antibody and complement-mediated lysis. An article by Vera et al. (see an article entitled “Tropomodulin-Binding Site Mapped to Residues 7-14 at the N-Terminal Hepta Repeats of Tropomyosin Isoform 5” by C. Vera, A. Sood, K. M. Gao, J. J. Lin and L. A. Sung in Arch. Biochem. Biophys., 378 pp. 16-24 (2000)) has described a monoclonal antibody which binds to the n-terminal region of tropomyosin, blocking its ability to form with troponin as part of the membrane complex. The binding site on tropomyosin that interacts with the troponin is residues 7-14, IEAVRKKV. The binding site on the tropomyosin that associates with the antibody is residues 4-10, ITTIEAV. Mimics to this region could initiate an autoimmune response.

There have been some molecular mimics suggested for MSH alpha as a cause for anorexia. (see, for example, an article entitled “Autoantibodies Against Appetite-Regulating Peptide Hormones and Neuropeptides: Putative Modulation by Gut Microflora” by S. O. Fetissov, S. M Hamze, M. Coeffer, et al., in Nutrition, 24 pp. 348-359 (2008)).

There are also some data involving proteolytic abnormalities in human anorexia and bulimia. Two proteases, dipeptidyl peptidase IV and prolyl endopeptidase, are abnormally low in anorexic and bulimic patients (see, for example, articles: “Lowered Serum Dipeptidyl Peptidase IV Activity in Patients with Anorexia and Bulimia Nervosa” by D. van West, P. Monteleone, A. DiLieto, I de Meister et al. in Eur. Arch. Psychiatry Clin. Neurosci., 250 pp. 86-92 (2000) and “Lower Serum Activity of Prolyl Endopeptidase in Anorexia and Bulimia Nervosa” by M. Maes, P. Monteleone, R. Bencivenga, F. Goossens, et al. in Psychoneuroendocrinolgy, 26 pp. 17-26 (2001)). Both enzymes are known to be involved in the modification or inactivation of hormones operating in the enteroinsular axis (see, for example, an article entitled “Eating Disorders: A Role for Dipeptidyl Peptidase IV in Nutritional Control” by M. Hildebrandt, M. Rose, H. Monnikes, W. Rutter, W. Keller and B. F. Klapp in Nutrition, 17 pp. 451-4 (2001)). Both proteases are involved in digestion of proline containing peptides (see, for example, see article entitled “Isolation and characterization of dipeptidyl peptidase IV from human placenta” by G. Puschel, R. Mentlein and E. Heymann in Eur. J. Biochem., 126 pp. 359-65 (1982)).

Therapeutic Approaches for Autoimmune Diseases: Over the last 50 years efforts have been undertaken to find treatments and cures for chronic autoimmune diseases. Most of this work has centered on finding general immunosuppressants.

Multiple Sclerosis (MS): MS is a chronic, slowly progressive disease of the central nervous system characterized pathologically by disseminated patches of demyelination in the brain and spinal cord, and clinically by multiple symptoms and signs, for example, paralysis, double vision, balance problems, and so forth, and by remissions and exacerbations.

People have tried to treat/cure MS using a number of different approaches. A first approach uses immunosuppressants. Examples include: ACTH (see, for example: an article entitled “Adrenal steroid therapy in neurological disease” by A. A. Eisen and J. W. Norris in Canad. Med. Ass. J., 100 pp. 27-30 (1969)); cyclophosphamide (see, for example, an article entitled “Intensive immunosuppression with cyclophosphamide in multiple sclerosis. Follow up of 110 patients for 2-6 years” by R. E. Gonsette, L. Demonty and P. Delmotte in J. Neurol., 214 pp 173-81 (1977)); prednisone (see, for example, an article entitled: “Prolonged effects of large-dose methylprednisolone infusion in multiple sclerosis” by J. L. Trotter and W. F. Garvey in Neurol., 30 pp. 702-8 (1980)); azothioprine (see, for example, an article entitled “Long-term treatment of multiple sclerosis with azathioprine” by W. R. Swinburn and L. A Liversedge in J. Neurol. Neurolsurg. Psychia., 36 pp. 124-126 (1973)); beta interferon (see, for example, an article entitled “Double blind study of intrathecal beta-interferon in multiple sclerosis: clinical and laboratory results” by C. Milanese, A. Salmaggi, L. La Mantia, A. Campi, M. Eoli, et al. in J. Neurol. Neurosurg. Psychia., 53 pp. 554-557 (1990)); interferon (see, for example, an article entitled “Multicentre double-blind study of effect of intrathecally administered natural human fibroblast interferon on exacerbations of multiple sclerosis” by L. Jacobs, A. M. Salazar, R. Herndon, P. A. Reese, et al. in Lancet, 2 pp. 1411-3 (1986)); and mitoxantrone (see, for example, an article entitled “Treatment of multiple sclerosis with mitoxantrone” by E. Mauch, H. H. Kornhuber, H. Krapf, U. Fetzer and H. Laufen et al. in Eur. Arch. Psychia Clin. Neurosci., 244 pp. 96-102 (1992)). Other procedures have been used to generally depress the immune system of multiple sclerosis patients. These include: plasmapheresis (see, for example, an article entitled “Plasmapheresis in multiple sclerosis: preliminary study” by H. L. Weiner and D. M. Dawson in Neurol., 30 pp. 1029-33 (1980)); use of antithymocyte globulin (see, for example, an article entitled “Multiple sclerosis treated with antithymocyte globulin—a five year follow-up” by L. K. Kastrukoff, D. R. McLean and T. A. McPherson in Can. J. Neurol. Sci., 5 pp. 175-8 (1978)); use of various antibody concoctions (see, for example, articles: “Immunogenic responses of progressive multiple sclerosis patients treated with an anti-T-Cell monoclonal antibody, anti-T12” by D. A. Hafler, R. J. Fallis, D. M Dawson, S. F. Scholssman et al in Neurol., 36 pp. 777-84 (1986); “An open trial of OKT3 in patients with multiple sclerosis” by B. G. Weinshenker, B. Bass, S. Karlik, G. C. Ebers and G. P. Rice in Neurol., 41 pp. 1047-52 (1991); and “Treating multiple sclerosis with monoclonal antibodies: a 2010 update” by M. Buttmann in Expert Rev. Neurother., 10 pp. 791-809 (2010)); use of a transfer factor (see, for example, an article entitled “A double blind trial of transfer factor vs. placebo in multiple sclerosis patients” by R. C. Collins, L. R. Espinoza, C. R. Plank, G. C. Ebers et al. in Clin. Exp. Immunol., 33 pp. 1-11 (1978)); and stem cell transplantation (see, for example, an article entitled “Stem cell transplantation in multiple sclerosis: current status and future prospects” by G. Martino, R. J Franklin, A. B. van Evercooren, and D. A. Kerr in Nat. Rev. Neurol., 6 pp. 247-55 (2010)). However, these human autoimmune diseases are chronic diseases. In many cases they have unpredictable relapsing and remitting episodes. Therefore, predicting when an immunosuppressive should be administered is questionable. In any event, the patient would be “exposed” to immunosuppressants throughout life. Therefore, the patient will have an increased susceptibility to any and all other infective agents.

Another approach is to eliminate the autoimmune response against the agent responsible, i.e., the mimic or immunogen, for the disease with a vaccine. This involves multiple injections of high concentrations of the immunogen, intending to deactivate the immune system against the immunogen—this is called antigen suppression. This approach has been used clinically in humans or in animals. An advantage of this approach is that the immune system is not totally immunosuppressed, but it is only suppressed against a particular autoimmune immunogen. A disadvantage is that one needs to identify precisely the immunogen. In most cases this is quite difficult. For example, four separate proteins have been identified as potential MS immunogens. Within these four proteins, several regions have been suggested as potential MS immunogens. Thus, there are potentially 20 different MS sites. This makes it difficult for this approach to succeed.

One of the proteins implicated in Multiple Sclerosis is the human myelin basic protein. Dr. Jonas Salk (see, for example, an article entitled “Myelin Basic Protein Treatment of MS” by J. Salk, F. Westall, J. Romine and W. Wiederholt in Neurol., 29 pp. 573 (1979)) attempted to cure multiple sclerosis with multiple injections of high concentrations of porcine myelin basic protein (MBP) and failed. When comparing the sequence of porcine MBP to the human MBP, one finds a major sequence change in one of the immunogenic regions. A similar therapy involves injection of a copolymer of four amino acids which represent four amino acids involved in the induction of allergic encephalomyelitis. Unfortunately, the co-polymer was modeled after an immunogen which, from the Salk experiment described above, was shown not to be not the MS immunogen. In fact, the co-polymer has been used with little success for over 20 years (see, for example, an article entitled “Glatiramer acetate and the glatiramoid class of immunomodulator drugs in multiple sclerosis: an update” by K. P. Joohnson in Expert Opin. Drug Metab. Toxicol., 6 pp 643-60 (2010)).

In other approaches, therapies have also been developed to alleviate symptoms rather than to eradicate the causes of the symptoms. These include: the use of brolitene (see, for example, an article entitled “A Trial of Brolitene in the Treatment of Spasticity” by G. D. Perkin and M. J. Aminoff in Br. J. Clin. Pharmac., 3 pp. 879-882 (1976)); the use of bactofen (see, for example, an article entitled “A double blind trial with bactofen and diazepam in spascity due to multiple sclerosis” by A. From and A. Heltberg in Acta Neurol. Scand., 51 pp. 158-66 (1975)); the use of carbamazeprine (see, for example, an article entitled “Treatment of paroxysmal disorders in multiple sclerosis with carbamazepine” by M. L. E. Espir and P. Millac in J. Neurol. Neurosurg. Psychiat., 33 pp. 528-531 (1970)), the use of glutethimide (see, for example, an article entitled “Glutethimide treatment of disabling action tremor in patients with multiple sclerosis and traumatic brain injury” by M Aisen, M Holzeer, M Rosen and M Dietz in Arch. Neurol., 48 pp. 513-5 (1991)); the use of 9-tetrahydrocannabinol (see, for example, an article entitled “Treatment of human spascity with delta 9-tetrahydrocannabinol” by D. J. Petro and C. Ellenberger, Jr. in J. Clin. Pharmacol., 21 pp. 413S-416S (1981)); the use of 4-aminopyridine (see, for example, an article entitled “4-aminopyridine in multiple sclerosis: prolonged administration” by D. Stefoski, F. A. Davis, W. E. Fitzsimmons, S. S. Luskin et al. in Neurol., 441 pp. 1344-8 (1991)); and the use of dantrolene (see, for example, an article entitled “The effects of dantrolene sodium in relation to blood levels in spastic patients after prolonged administration” by W. J. Meyler, H. Bakker, J. J. Kok, S. Agoston and H. Wesseling in J. Neurol. Neurolsurg. Psychia., 44 pp. 334-339 (1981)).

Further approaches include: use of hyperbaric oxygen (see, for example, an article entitled “Hyperbaric oxygen in multiple sclerosis: a double blind trial” by C. M. Wiles, C. R. A. Clarke, H. P. Irwin, E. F. Edgar and A. V. Swan in Brit. Med. J., 292 pp. 367-371 (1986)); linoelate supplementation (see, for example, an article entitled “Double blind trial of linoleate supplementation of the diet of multiple sclerosis” by J. H. D. Millar, K. J. Zilkha, M. J. S. Langman, H. P. Wright, et al. in Brit. Med. J., 1 pp. 765-768 (1973)); and the use of diets (see, for example, articles: “Letter: Gluten free diet as treatment for multiple sclerosis” F. H. Hafner in Postgrad. Med., 59 p. 20 (1976); “Review of MS patient survival on a Swank low saturated fat diet” by R. L. Swank and J. Goodwin in Nutrition, 19 pp. 161-2 (2003); and “A double blind controlled trial of long chain n-3 polyunsaturated fatty acids in the treatment of multiple sclerosis” by D. Bates, N. E. F. Cartlidge, J. M. French, M. J. Jackson, et al. in J. Neurol. Neurolsurg. Psycia., 52 pp. 18-22 (1989)). These approaches have provided little long term success.

Lastly, in another approach, O. A. Mlalovyts'ka reports a therapy entailing the use of various enzymes (see the abstract of an article (in Ukrainian) entitled “Effect of phlogenzym in long-term treatment of patients with multiple sclerosis” by O. A. Mlalovyts'ka in Lik. Sprava., pp. 109-13 (April-June, 2003)). The abstract indicates that Phlogenzym, a product comprised of the enzymes bromelain and trypsin and the anti-oxidant rutin were administered orally to 74 patients with progressing MS. The abstract indicates that over a one to three year period, their symptoms gradually improved and the rate of progression slowed.

Myasthenia Gravis: Myasthenia gravis is characterized by progressive fatigability of muscles due to impairment of conduction at the myoneural junction. Patients complain of double vision, difficulty swallowing, chewing and talking. The course is variable and there may be prolonged remissions.

People have tried to treat/cure Myasthenia Gravis using immunosuppressants such as corticosteroids that reduce inflammatory signaling molecules and adhesion molecules and reduce the transport of inflammatory cells. For example, prednisone has been used (see, for example, an article entitled “Treatment of autoimmune myasthenia gravis” by D. Richman and M. Agius in Neurology, 61 pp. 1652-1661 (2003)). In general, this approach induces remission in many patients, but long-term usage has serious side-effects, and does not cure the disease.

Uveoretinitis: Uveoretinitis encompasses a collection of human sight-threatening inflammatory eye diseases of autoimmune etiology. Experimental uveoretinitis is the model for the experimental model for these diseases.

The use of immunosuppressants is a standard procedure for treating uveoretinitis. Such procedures include: use of adalimumab (see, for example, an article entitled “Adalimumab therapy for refractory uveitis: a pilot study” by M. S. Diaz-Liopis, S. Garia-Delpech, D. Salom, P. Udaondo, et al. J. Ocul. Pharmacol. Ther., 24 pp. 351-61 (2008)); use of dexamethasone (see, for example, an article entitled “Dexamethasone sustained drug delivery implant for the treatment of severe uveitis” by G. J. Jaffe, P. A. Pearson and P. Ashton in Retina 20 pp. 402-3 (2000)); use of guanethidine (see, for example, an article entitled “Guanethidine in the therapy of glaucoma” by J. A. Castren, S. Pohjola, P. Pakarinen and K. Karjalainen in Ophthalmologica, 155 pp. 194-204 1968); use of infliximab (see, for example, an article entitled “Treatment of refractory posterior uveitis with infliximab: a 7 year follow study” by R. Lopez-Gonzalez, E. Loza, J. A. Jover, J. M. Benitez del Catillo, et al. in Scand. J. Rheumatol., 38 pp. 58-62 (2009)); and use of cyclosporine (see, for example, an article entitled “Cyclosporine therapy for uveitis: long-term follow up” by R. B. Nussenblatt, A. G. Palestine and C. C. Chan in J. Ocul. Pharmacol., 1(4) pp. 369-82 (1985)). Oral administration of retinal antigens has also been used to attempt specific antigen suppression (see, for example, an article entitled “Treatment of uveitis by oral administration of retinal antigens: results of a phase I/II randomized masked trial.” by R. B. Nussenblatt, I. Gery, H. L. Weiner, F. L. Ferris, J. Shiloach, et al. in Am. J. Ophthalmol., 123 pp. 583-92 (1997)). The use of interferon alpha 2a (see, for example, an article entitled “Interferon alpha-2a: a new treatment option for long lasting refractory cystoid macular edema in uveitis? A pilot study” by C. M. Deuter, I. Koetter, I. Guenaydin, N. Stuebiger and M. Zierhut in Retina 26 pp. 786-91 (2006)) controls various secondary problems associated with the disease, including macular edema. Another therapy entails cryotherapy (see, for example, an article entitled “Treatment of peripheral uveoretintitis by cryotherapy” by T. M. Aaberg, T. J. Cesarz and R. R. Flickinger in Am. J. Ophthalmol., 75 pp. 685-8 (1973)). In addition, the use of certain immunomodulator drugs has been proposed (see, for example, an article entitled “The potential of newer immunomodulating drugs in the treatment of uveitis: a review” by J. Salzmann, and S. Lightman in BioDrugs, 13 pp. 397-408 (2000)).

Lupus Erythematosus: Lupus erythematosus is a collection of chronic systemic autoimmune diseases—four main types of lupus exist. Of these, systemic lupus erythematosus is the most serious. Symptoms vary from person to person. Almost all people with systemic lupus erythematosus have joint pain and swelling—some develop arthritis. Other major symptoms include: fatigue, fever, mouth sores, hair loss, sensitivity to light, skin rashes and chest pains.

People have tried to treat/cure Lupus erythematosus using a number of different approaches. A first approach entails supplementation of beneficial bacteria, known as probiotics, to compete with the effects of suspected harmful bacteria, such as Lactobacillus bulgaricus delbruekii and Streptococcus thermophilus (see U.S. Pat. No. 6,696,057 entitled “Composition and method for treatment of gastrointestinal disorders and hyperlipidemia”). Use of this approach has been intended to reduce symptoms associated with the disease, but there have been no data published to support this.

Another approach entails delivering human recombinant Dnase 1 intravenously or subcutaneously. Human recombinant Dnase 1 is an enzyme that breaks down excess DNA fragments found in serum (see, for example, a book entitled “Dubios' lupus erythematosus,” by D. Wallace, B. Hahn and E. Dubois at p. 1354, Lippincott Williams & Wilkins (2007)). In general, this approach has not been successful at treating or curing the disease.

Ulcerative Colitis (UC): UC is a chronic, nonspecific, inflammatory and ulcerative disease of the colon which is characterized by the passage of bloody and purulent mucus, either alone or accompanying formed or watery stools. The cause of UC is unknown.

People have tried to treat/cure UC using a number of different approaches. A first approach entails giving patients antibiotics to kill microbes that may be causing or exacerbating symptoms. For example, the following antibiotics have been used: clarithromycin, ethambutol, metronidazole, and rifabutin (see, for example, an article entitled “Treatment of Ulcerative Colitis Using Fecal Bacteriotherapy” by T. Borody, E. Warren, S. Leis, R. Surace and O. Ashman in J. Clin. Gastroenterol., 37(1) pp. 42-47 (2003)). Although antibiotics can help treat the symptoms of ulcerative colitis, in general, this approach has not been successful at curing the disease. A second approach entails treating patients with various immunosuppressants such as azothioprine, prednisone, prednisolone sodium phosphate enema, mercaptopurine, cyclosporine, and clofazimine (see, for example, an article entitled “Treatment of Ulcerative Colitis Using Fecal Bacteriotherapy” by T. Borody, E. Warren, S. Leis, R. Surace and O. Ashman in J. Clin. Gastroenterol., 37(1) pp. 42-47 (2003)). Although these drugs reduce symptoms, they have significant side effects for long-term use. Another approach entails treating patients with various anti-Tumor Necrosis Factor antibodies such as infliximab (see, for example, an article entitled “Review article: Infliximab therapy for inflammatory bowel disease—seven years on” by P. Rutgeerts, G. Van Assche, and S. Vermeire in Aliment Pharmacol. Ther., 23(4) pp. 451-63 (Feb. 15, 2006)). However, not all people respond to anti-Tumor Necrosis Factor antibodies, some have allergic reactions, and their use increases the risk of infection. Another approach suppresses the immune system by suppressing inflammatory signaling molecules, thereby slowing down production of immune cells, using anti-tumor necrosis factor drugs to block TNF activity. For example, the following have used: clarithromycin, ethambutol, metronidazole, mercaptopurine, and rifabutin (see, for example, an article entitled “Treatment of Ulcerative Colitis Using Fecal Bacteriotherapy” by T. Borody, E. Warren, S. Leis, R. Surace and O. Ashman in J. Clin. Gastroenterol., 37(1) pp. 42-47 (2003)). In general, this approach can treat with the increased risk of infection and other side-effects, but not cure the disease.

Another approach entails treating with synthesized anti-oxidant chemicals to block free-radicals from damaging gastrointestinal tissues. These chemicals, called aminosalicylates, include mesalamine, olsalazine and salazopyrin (see, for example, an article entitled “Treatment of Ulcerative Colitis Using Fecal Bacteriotherapy” by T. Borody, E. Warren, S. Leis, R. Surace and O, Ashman in J. Clin. Gastroenterol., 37(1) pp. 42-47 (2003)). In general, this approach treats symptoms of some patients but does not, in general, cure the disease. Another approach entails treating with various anti-motility drugs such as loperamide hydrochloride and codeine phosphate to reduce diarrhea (see, for example, an article entitled “Treatment of Ulcerative Colitis Using Fecal Bacteriotherapy” by T. Borody, E. Warren, S. Leis, R. Surace, and O. Ashman in J Clin Gastroenterol 37(1) pp. 42-47 (2003)). In general, this reduces the frequency of stools, but it has a risk of inducing toxic megacolon associated with the disease. Another approach entails treating with various enzymes extracted from plants, such as bromelain (see, for example, an article entitled “Use of Bromelain for Mild Ulcerative Colitis” by S. Kane and M. Goldberg in Annals of Internal Medicine, vol. 132, no. 8 p. 680 (Apr. 18, 2000)). Although the article states “Orally administered bromelain was anecdotally reported to induce clinical and endoscopic remission of ulcerative colitis in two patients whose disease was refractory to multi-agent conventional medical therapy,” in general, this approach has not been successful at treating or curing the disease in significant numbers of patients.

Another approach entails treating with short chain fatty acids such as butyric acid in enemas to rapidly nourish colonic tissues (see an article entitled “Local Short-Chain Fatty Acids Supplementation without Beneficial Effect on Inflammation in Excluded Rectum” by J. Schauber, T. Bark, E. Jaramillo, M. Katouli, B. Sandstedt and T. Svenberg in Scand. J. Gastroenterol., (2) (2000)). In general, this approach has not been successful at treating or curing the disease. Another approach entails treating with bismuth compounds such as oral bismuth subsalicylate to chemically remove toxic hydrogen sulfide from the intestine (see, for example, an article entitled “Production and elimination of sulfur-containing gases in the rat colon” by F. Suarez, J. Fume, J. Springfield and M. Levitt in Am. J. Physiol., 274(4 Pt 1): pp. G727-33 (April 1998)). In general, this approach has not been successful at treating or curing the disease. Another approach entails treating with anti-inflammatory compounds from plants such as aloe vera gel (see, for example, an article entitled “Randomized, double-blind, placebo controlled trial of aloe vera gel for active ulcerative colitis” by L. Langmead, R. Feakins, S. Goldthorpe, H. Holt, E. Tsironi, A. De Silva, D. Jewell and D. Rampton in Aliment Pharmacol. Ther., 19 pp. 739-747 (2004)). Although this approach has been able to reduce symptoms, the results have not shown to be successful at treating or curing the disease in large numbers of patients over long periods of time.

Another approach entails treating by supplementing the diet with substances to promote the growth of beneficial bacteria, called prebiotics, such as germinated barley foodstuff (see, for example, an article entitled “The Dietary Combination of Germinated Barley Foodstuff plus Clostridium butyricum Suppresses the Dextran Sulfate Sodium-Induced Experimental Colitis in Rats” by Y. Araki, Y. Fujiyama, A. Ando, S. Koyama, O. Kanauchi and T. Bamba in Scand. J. Gastroenterol., 35 (10) pp. 1060-7 (October 2000)). In general, this approach has been successful at helping patients go into, and maintain, remission longer than a placebo. However, it has not been shown to stop periodic flare-ups associated with the disease. Another approach entails treating by infecting the patient with intestinal worms to change the immune behavior from Th1 to Th2 which reduces inflammation in the colon (see, for example, an article entitled “What is the origin of ulcerative colitis? Still more questions than answers” by M. Lukas, M. Bortilik and Z. Maratka in Postgrad. Med. J., 82 pp. 620-625 (2006)). In general, this approach has helped treat patients' symptoms, but does not cure the disease. Another approach entails treating by supplementing the diet with colonically delivered phosphatidylcholine which is believed to make the colon hydrophobic and less vulnerable to toxins (see, for example, an article entitled “Retarded release phosphatidylcholine benefits patients with chronic active ulcerative colitis” by W. Stremmel, U. Merle, A. Zahn, F. Autschbach, U. Hinz and R. Ehehalt in Gut, 54 pp. 966-971 (2005)). Although this approach helped many patients achieve remission, a significant number complained of side-effects, bloating and nausea in a later study.

Another approach entails treating by supplementation of beneficial bacteria, known as probiotics, to compete with the effects of suspected harmful bacteria, such as Escherichia coli Nissle 1917 (see, for example, an article entitled “Bacteria as the cause of ulcerative colitis” by M. Campieri and P. Gionchetti in Gut, 48(1) pp. 132-5 (January 2001)). Although this approach has been shown to reduce symptoms and lengthen the time between flare-ups of the disease, it could not prevent periodic flares that occur about once a year in large numbers of people. Another approach entails treating by replacing bacteria in the gastrointestinal tract with a human fecal transplant (see, for example, an article entitled “Treatment of Ulcerative Colitis Using Fecal Bacteriotherapy” by T. Borody, E. Warren, Sh. Leis, R. Surace and O. Ashman in J. Clin. Gastroenterol., 37(1) pp. 42-47 (2003)). In general, although this approach helped mitigate patients' symptoms, it has not been successful at curing the disease in all patients.

Celiac Disease (CD): CD is a fairly common genetically determined inflammatory disease of the intestine, and is induced by ingestion of gluten and related proteins from barley, oats and rye. Symptoms are age dependent. Children suffer from diarrhea, fatigue and weight loss; in older individuals symptoms are more diffuse. Both immune responses to gluten and autoimmune triggers are thought to be involved in the disease. In active CD, the small intestinal mucosa is affected by both innate and adaptive immune processes. Inflammatory lesions occur in the upper small intestinal mucosa characterized by increased numbers of intraepithelial lymphocytes together with small intestinal villous atrophy and crypt hyperplasia.

People have tried to treat/cure CD with various enzymes extracted from plants and microbes such as a cysteine endoprotease from germinating barley and prolyl endopeptidase from Sphingomonas capsulata (see, for example, an article entitled “The effects of ALV003 pre-digestion of gluten on immune response and symptoms in celiac disease in vivo” by J. Tye-Din, R. Anderson, R. French, G. Brown, P. Hodsman, M. Siegel, W. Botwick and R. Shreeniwas in Clinical Immunology, vol. 134 pp. 289-295 (2010)). Although the article states that the enzymes were successful at destroying targeted gluten peptides and prevented an immune response to gliadin or 33 mer, the patients' symptoms did not improve and even worsened during the clinical trial. This indicates that, in general, the approach has not been successful at treating or curing the disease in significant numbers of patients.

Anorexia nervosa: Anorexia nervosa is an eating disorder characterized by refusal to maintain a healthy body weight and an obsessive fear of gaining weight. People have tried to treat/cure anorexia nervosa with antidepressants such as olanzapine (see, for example, an article entitled “Review of Medication Use for Children and Adolescents with Eating Disorders” by J. Couturier and J. Lock in J. Can. Acad. Child. Adolesc. Psychiatry, 16:4 (November 2007)). In general, this approach can treat symptoms but does not cure the disease.

Rheumatoid Arthritis: Rheumatoid arthritis is an autoimmune disease characterized by chronic inflammation of the synovial membrane, resulting in destruction of cartilage and bone in affected joints. People have tried to treat/cure rheumatoid arthritis using a number of different approaches. A first approach entails giving patients antibiotics to kill microbes that may be causing or exacerbating symptoms. For example, the antibiotic minocyclin has been used (see, for example, an article entitled “Use of aminocycline in rheumatoid arthritis: a district general hospital experience” by E. Suresh, I. M. Morris and P. C. Mattingly in Ann. Rheum. Dis., 63 pp. 1354-1355 (2004)). Although the treatment helped some patients reduce their symptoms, many had substantial side effects and had to stop treatment. In general, this approach has not been successful at curing the disease. A second approach entails giving patients various immunosuppressants such as prednisone, methotrexate, infliximab, adalimumab and entanercept. (see, for example, an article entitled “Serious infections with antirheumatic therapy: are biologicals worse?” by K. L Winthrop in Ann. Rheum. Dis., 65(Suppl III) pp. iii54-iii57 (2006)). Although these chemicals are sometimes effective at reducing symptoms, they have significant side-effects for long term use. In general, this approach has been successful at treating, with risk of infection, but not at curing the disease. Another approach entails the use of various enzymes (see, for example, an article entitled “Basic studies on enzyme therapy of immune complex diseases” by C. Steffen and J. Menzel in Wein. Klin. Wochenschr., 97(8) pp. 376-85 (Apr. 12, 1985)). The abstract indicates that several unidentified enzymes were administered orally to rabbits whose arthritis was induced experimentally with antigens and that the enzymes helped reduce symptoms. Because this was an animal study, there is no data to indicate if the approach can be successful at treating or curing the disease in significant numbers of patients.

Autoimmune Autistic Spectrum Disorder: There are many causes of autistic spectrum disorders and research has shown that a history of autoimmunity, especially Celiac disease, ulcerative colitis, and rheumatoid arthritis in the mother are risk factors for the child to become diagnosed with an autistic spectrum disorder. The causes and treatments for autistic spectrum disorder people are highly varied. Some are sensitive to gluten and casein and respond to dietary elimination of gluten and casein; some respond to antibiotics, such as oral vancomyocin, but their symptoms return soon after stopping consumption of the antibiotic; some respond to dietary supplementation with probiotics as well as enzymes. One doctor announced that he had observed favorable responses with an enzyme sold under the trade name Nattokinase. Lactobacilli, Bifidobacterium, Lactococcus, and Saccharomyces boulardii are probiotics that are often recommended by doctors to autistic children who have gastrointestinal issues such as diarrhea or constipation.

### SUMMARY

One or more embodiments of the present invention comprise destroying or deactivating an immunogen, mimic and/or antigen for a disease before it encounters the immune system. In particular, an embodiment of one method comprises administering a source of a protease that destroys or deactivates an immunogen, mimic and/or antigen specific to a particular autoimmune disease before it encounters the immune system.

### DETAILED DESCRIPTION

One or more embodiments of the invention are methods for delivering or administering a medicament comprised of protolytic enzymes, as chemical compounds, or for delivering or administering a medicament comprised of probiotic microorganisms that produce such protolytic enzymes, which protolytic enzymes eradicate or degrade autoimmune mimics to prevent the interaction of the autoimmune mimics with a host's (otherwise referred to herein as a patient) patient immune system—such hosts being mammals such as, for example and without limitation, humans, dogs, cats and so forth. As used herein, autoimmune mimics are structurally comparable to autoimmune immunogens found in tissues of hosts afflicted with an autoimmune disease. In accordance with one or more such embodiments, the proteolytic enzymes are delivered to the gastrointesintal tract by probiotic microorganisms, by a drug delivery system, or by a combination of enzymic chemistries and probiotic microorganisms. As used herein, the term host also refers to a patient.

In accordance with one or more embodiments, the proteolytic enzymes include selected proteases which can be delivered: (a) as pure chemicals; or (b) as pure chemicals in combination with live and/or dead microorganisms or portions of such microorganisms. In accordance with one or more such embodiments, the selected protease(s) will reduce the concentrations of peptides in a host's gut which are composed of more than six (6) amino acids. As such, the selected protease(s) will reduce the concentration of potential autoimmune mimics already present in a host's inflicted gut, and prevent new autoimmune mimics from being produced. Advantageously, as a result, the probiotic microorganisms and/or proteases will eliminate potential autoimmune mimics to provide a procedure for treating/curing chronic autoimmune diseases.

In accordance with one or more embodiments, a medicament comprised of probiotic microorganisms or proteases or a combination of probiotic microorganisms and proteases may be delivered or administered orally, by suppository, or by injection into a patient's gut (for example, and without limitation, enema, endoscope, colonoscope, robotically actuated capsule, and so forth). In accordance with one or more such embodiments, treatment would range from about weekly to about daily, and be ongoing until symptoms of the disease have disappeared.

Multiple Sclerosis (MS): In accordance with one or more embodiments of the present invention, a medicament comprised of an effective dose or amount of subtilisin is administered to a patient to treat/cure MS. An effective dose or amount of subtilisin is a dose or amount of the protease that is effective in destroying or deactivating mimics, immunogens and/or antigens that cause or exacerbate MS. The effective amount of subtilisin administered will depend upon the severity of the disease process, but will range between about 2,000 fibrinolytic units/day and about 10,000 fibrinolytic units/day. For example, the subtilisin will be administered in one or more, preferably three, doses daily of subtilisin. It is believed that hydrolytic processes of the subtilisin will act in two areas: the gut and the blood. In the gut, the subtilisin will destroy or deactivate potential MS mimics before they can reach a sufficient level for immune activation, and, in the blood the subtilisin will destroy or deactivate any MS immunogens and antigens that are released from central nervous system myelin which could interact with the immune system and prolong a clinical exacerbation.

In accordance with one or more such embodiments of the present invention, a medicament comprised of an effective amount of a non-pathogenic microorganism and/or its spore (that are capable of providing subtilisin), is administered to a patient to treat/cure MS. In accordance with one or more such embodiments, suitable microorganisms and spores include, for example, but not limited to, Bacillus subtilis, Bacillus licheniformis, and Bacillus lentus and their various strains. An effective amount of the microorganism and/or its spores is an amount that is effective to cause destruction or deactivation of mimics, immunogens and/or antigens that cause or exacerbate MS. In accordance with one or more such embodiments, an effective amount is from about 100 thousand CFU to about 600 billion CFU per dose (CFU designates colony forming units), where the dose is administered about one or more times per week, or as often as about one to about three times daily.

In accordance with one or more embodiments of the present invention, a medicament comprised of an effective amount of parts of, or entire broken up, microorganisms and/or their spores capable of providing subtilisin, is administered to a patient to treat/cure MS. Parts of the microorganisms and/or spores can be separated and selected, using any one of a number of methods that are well known to those of ordinary skill in the art, for their bioactive properties to help ensure and improve the rate of the destruction or deactivation of mimics, immunogens or immunogens that cause or exacerbate MS and/or to improve the effectiveness of enzymes in the gastrointestinal or respiratory tract in destroying or deactivating such mimics, immunogens and/or antigens. As used herein, the term gastrointestinal tract includes the oral cavity. In accordance with one or more embodiments, the parts of the microorganisms or spores selected can be, but are not limited to, adjuvants such as muramyl dipeptide. An adjuvant's content can be increased or decreased using any one of a number of methods that are well known to those of ordinary skill in the art to saturate or starve the gastrointestinal tract or respiratory tract of the adjuvant to prevent an optimum ratio of adjuvant to mimic from being close enough to one that initiates and/or maintains the autoimmune reaction. In accordance with one or more embodiments, suitable microorganisms and spores are, but are not limited to, Bacillus subtilis, Bacillus licheniformis, and Bacillus lentus. An effective amount of the parts of, or entire broken up, microorganisms and/or their spores is an amount sufficient to cause destruction or deactivation of mimics, immunogens and/or antigens that cause or exacerbate MS. In accordance with one or more such embodiments, an effective amount of parts or entire broken up microorganisms is from about 100 thousand CFU to about 600 billion CFU per dose, where the dose is administered about one or more times per week, or as often as about one to about three times daily. Methods for breaking up suitable microorganisms and/or spores include, for example and without limitation, sonication, crushing, shearing, oxidation, exposure to photonic radiation or chemical (for example, not limited to, enzymic) cleavage of the microorganisms.

In accordance with one or more embodiments of the present invention, an above-described medicaments can be administered orally, where an oral delivery mechanism includes, for example and without limitation, a capsule; a tablet; a spray; and a drink, food, powder, gum, candy, gel, or cream containing the product. The term administered orally includes sublingually, and on an absorbent substrate or adsorbent substrate.

In one or more embodiments of the present invention, an above-described medicament can be administered rectally, where a rectal delivery mechanism includes, for example and without limitation, an enema, a fecal transplant, a gel, a cream, an ointment, or a suppository.

In accordance with one or more embodiment, a fecal transplant includes at least some of the following. First, a donor of feces is screened to look for parasites, pathogenic microorganisms, and to measure the kinds of microbes that are in the donor's feces. The donor's feces are also analyzed for chemicals having, for example, but not limited to, proteolytic activity. Next, the microbial and chemical measurements are compared against a set of requirements for a successful transplant, for example, but not limited to, the presence of bacteria or chemical activity that can destroy mimics, immunogens and/or antigens that cause or exacerbate MS. Next, the donor's feces may be corrected for pH level by adding acids, bases, or appropriate buffering agents. Any imbalance of enzymes may be corrected by selecting an appropriate enzyme or pro- or co-enzyme producing microbe. A candidate microbe may be identified in the manner described below. Next, undesirable bacteria can be neutralized or killed. If the donor's feces do not have sufficient ability to destroy mimics, immunogens and/or antigens that cause or exacerbate MS, then microorganisms that are capable of destroying mimics, immunogens and/or antigens that cause or exacerbate MS, are added, in an effective amount, to the donor's feces prior to a fecal enema or around the time of the fecal enema to populate the sick person's gastrointestinal tract.

In accordance with one or more embodiments of the present invention, enzymes for destroying or inactivating mimics, immunogens and/or antigens are delivered to a patient. To find and select microorganisms as sources of such enzymes, one can search the protein database maintained by the National Institute of Health (“NIH”) at http://www.ncbi.nlm.nih.gov/protein. The following is an example of how to identify microorganisms as sources for candidate enzymes. Enter search terms such as, for example and without limitation, “prolyl endopeptidase,” “prolyl exopeptidase,” “prolyl oligopeptidase,” “subtilisin-like serine protease,” and/or “prolyl oligopeptidase-like” for enzymes that can cleave a mimic, immunogen and/or antigen at a proline active site. The protein search engine will return a list of microorganisms that can make such enzymes. Identifying microorganisms as sources for enzymes such as, for example and without limitation, a serine protease, entails entering search terms such as, for example and without limitation, “serine endopeptidase” and/or “serine exopeptidase” and/or “serine oligopeptidase.” Then, to make sufficient quantities of a particular enzyme, the microorganisms identified as a result of, but not limited to, the above-described database search can be grown using any one of a number of methods that are well known to those of ordinary skill in the art, and the enzyme can be extracted from them using any one of a number of methods that are well known to those of ordinary skill in the art. Alternatively, the relevant gene that expresses the enzyme within the microorganism capable of making the enzyme can be determined using any one of a number of methods that are well known to those of ordinary skill in the art, and the relevant gene can be inserted into another microorganism using any one of a number of methods that are well known to those of ordinary skill in the art to make the enzyme for extraction. After extraction using any one of a number of methods that are well known to those of ordinary skill in the art, and followed by purification steps using any one of a number of methods that are well known to those of ordinary skill in the art, if needed, the relevant enzyme can be used in accordance with one or more embodiments of the present invention.

In accordance with one or more embodiments, the medicament can be administered transdermaly, where a transdermal delivery mechanism includes, for example and without limitation, a skin patch, a spray, a gel, a cream, an ointment or a bath.

In accordance with one or more embodiments, the medicament can be administered intravenously, where an intravenous delivery mechanism includes, for example and without limitation, injection of the medicament mixed into an intravenous solution.

In accordance with one or more embodiments, the medicament can be administered by inhalation, where an intravenous delivery mechanism includes, for example and without limitation, a nebulized powder inhaled by the nose or mouth.

In accordance with one or more embodiments, a medicament comprised of subtilisin can be combined with other enzymes or probiotics such as, for example and without limitation, PepO or Lactobacillus lactis L1A.

Subtilisin, a serine protease or serine proteolytic enzyme, can degrade one or more mimics of the three chemically defined MS peptide sequences: tryptophan peptide from myelin basic protein (see, for example, an article entitled “Essential Chemical Requirements for Induction of Allergic Encephalomyelitis” by F. C. Westall, A. B. Robinson, J. Caccam, J. J. Jackson and E. H. Eylar in Nature, 229 pp. 22-24 (1971)); PHE SER TRP GLY ALA GLU GLY GLN ARG, mid region from myelin basic protein (see, for example, an article entitled “Biological Activity and Synthesis of an Encephalitogenic Determinant” by R. F. Shapira, C. H. Chou, S. Mc Kneally, E. Urban and R. F. Kibler in Science, 172 pp. 736-738 (1971)); THR THR HIS TYR GLY SER LEU PRO GLN LYS, and the hyperacute site from myelin basic protein (see, for example, an article entitled “Hyperacute Autoimmune Encephalomyelitis-Unique Determinant Conferred by Serine in a Synthetic Autoantigen” by F. C. Westall, M. Thompson and V. A. Lennon in Nature, 269 pp. 425-427 (1977)) PRO GLN LYS SER GLN ARG THR GLN ASP GLU ASN PRO VAL. Subtilisin enzymes that are useful for treating MS are subtilisins from any of the subtilisin proteases included in the subtilase superfamily, family subtilisin, and subgroup true subtilisins. For example, one or more embodiments of the present invention comprise administering subtilisin proteases in the “true subtilisin” subgroup which includes subtilisin BPN′, subtilisin Carlsberg and subtilisin NAT.

Serine endo- and exopeptidases are of widespread occurrence and diverse function. Distinct families of serine proteases are divided into six (6) superfamilies (see, for example, an article entitled “Families of Serine Proteases” by N. D. Rawlings and A. J. Barrett in Methods of Enzymology, 244 pp. 19-61 (1994)). The two largest superfamilies are the chymotrypsin-like and the subtilisin-like (subtilase) superfamilies. These two clans are distinguished by a highly similar arrangement of catalytic His, Asp, and Ser residues in radically different beta/beta (chymotrypsin) and alpha/beta (subtilisin) protein scaffolds. If pairwise sequence identity within the catalytic domains is plotted graphically for all members of the subtilase superfamily, clustering occurs into groups or families in which members show higher sequence identity to each other. There are six (6) families of which one is the “subtilisin” family. It includes mainly enzymes from Bacillus, with subgroups of “true subtilisins” (>64% identity), high-alkaline proteases (>55% identity), and intracellular proteases (>37% identity) (see, for example, an article entitled “Subtilases: The Superfamily of Subtilisin-Like Serine Proteases” by R. J. Siezen and J. A. M. Leunissen in Protein Sci., 6 pp. 501-523 (1997).)

In accordance with one or more embodiments of the present invention, subtilisins used in the therapeutic treatment of MS includes any of the subtilisin proteases included in the superfamily subtilase, family subtilisin, and subgroup true subtilisins. The definition the requirements of this group are set forth in an article entitled “Subtilases: The Superfamily of Subtilisin-Like Serine Proteases” by R. J. Siezen and J. A. M. Leunissen in Protein Sci., 6 pp. 501-523 (1997), which article is incorporated herein by reference in its entirety.

Myasthenia Gravis (MG): In accordance with one or more embodiments of the present invention, a medicament comprised of an effective dose of oligopeptidase F (PepF) is administered to a patient to treat/cure MG. PepF belongs to the M3 metalloprotease family. While most bacterial PepFs are cytoplasmic endopeptidases, some are secreted, for example, the enzyme from Bacillus amyuloliquefaciens. Pep F has been seen in a variety of bacterial genuses including, Lactococcus and Bacillus and in Bacillus subtilis. (see, for example, the following articles: “Biochemical and Genetic Characterization of PepF, an Oligopeptidase from Lactococcus Lactis” by V. Monnet, M. Nardi, A. Chopin, M.-C. Chopin and J.-C. Gripon in J. Biol. Chem., 269 pp. 32070-32076 (1994); “Duplication of the PepF Gene and Shuffling of DNA Fragments on the Lactose Plasmid of Lactococcus Lactis” by M. Nardl, P. Renault and V. Monnet in J. Bacteriol., 179 pp. 4164-4171 (1997); “Overexpression of the PepF Oligopeptidase Inhibots Sporulation Initiation in Bacillus Subtilis” by K. Kanamaru, K. S. Stephenson and M. Perego in J. Bacteriol., 184 pp. 43-50 (2002); and “Characterization of a novel Pep-F-like oligopeptidase secreted by Bacillus amyloliquefaciens” by S.-H Chao, T.-H. Cheng, C.-Y. Shaw, M. H. Lee, Y.-H. Hsu and Y.-C. Tsai in 23-7A. App. Environ. Microbiol., 72 pp. 968-971 (2006)). Pep F favors breaking bonds n-terminal to hydrophobic residues, but also can break bonds between proline and hydrophobic residues. Furthermore, it has been shown to break bonds n-terminal to charged amino acids. With respect to alpha MSH, this protease should break those bonds of peptides that mimic the c-terminal of alpha MSH. In accordance with one or more such embodiments, an effective dose or amount of PepF is a dose or amount of the protease (for example, in sufficient concentration) that is effective in destroying or deactivating mimics, immunogens antigens that cause or exacerbate MG. The effective amount of endopeptidase F (PepF) administered will depend upon the severity of the disease process (the medicament will be administered one or more, preferably three, doses daily), but will range between about 20 units/day and about 200 units/day where the definition of a unit is an amount of PepF required to cleave one micromole of bradykinin at a pH of 8.0 and a temperature of 40° C.

In accordance with one or more further embodiments, a medicament comprised of an effective dose of endopeptidase O2 (PepO2) is administered to a patient to treat/cure MG. In accordance with one or more such embodiments, an effective dose or amount of PepO2 is a dose or amount of the protease (for example, in sufficient concentration) that is effective in destroying or deactivating mimics, immunogens antigens that cause or exacerbate MG. The effective amount of endopeptidase O2 (PepO2) administered will depend upon the severity of the disease process (the medicament will be administered one or more, preferably three, doses daily), but will range between about 20 units/day and about 200 units/day where the definition of a unit is an amount of PepO2 required to cleave one micromole of BCN (f193-209) at a pH of 6.5 and a temperature of 25° C. While endopeptidase O2 retains an ability to break bonds n-terminal to hydrophobic residues, the added specificity for post-proline bonds distinguishes it from other PepO-type endopeptidases (see, for example, an article entitled “Identification and Characterization of Lactobacillus Helveticus PepO2, an Endopeptidase with Post-Proline Specificity” by Y.-S. Chen, J. E. Christensen, J. R. Broadbent and J. L. Steele in Appl. Environ. Microbiol., 69 pp. 1276-1282 (2003)).

In accordance with one or more further embodiments of the present invention, a medicament comprised of an effective amount of a non-pathogenic microorganism and/or its spores (that are capable of providing PepF) is administered to a patient to treat/cure MG. In accordance with one or more such embodiments, the microorganism and spores include, for example and without limitation, Lactobacillus jensenii, Lactobacillus crispatus, Lactobacillus johnsonii, Lactobacillus plantarum, Lactobacillus helveticus, Lactobacillus amylolyticus, Lactobacillus salivarius, Lactobacillus ultunensis, Lactobacillus rhamnosus, Lactobacillus acidophilus, Lactobacillus delbrueki bulgaricus, Lactobacillus gasseri, Lactobacillus casei, Lactobacillus coleohominis, Lactobacillus fermentum, Lactobacillus paracasei, Lactococcus lactis cremoris, Enterococcus faecalis, Bacillus cereus (spore-forming), Campylobacter subtilisis, and Oenococcus oeni and their various strains. An effective amount of the microorganism and/or its spores is an amount that is effective to cause destruction or deactivation of mimics, immunogens and/or antigens that cause or exacerbate MG. In accordance with one or more such embodiments, an effective amount is from about 100 thousand CFU to about 600 billion CFU per dose, where the dose is administered about one or more times per week, or as often as about one to about three times daily.

In accordance with one or more embodiments of the present invention, a medicament comprised of an effective amount of a non-pathenogenic microorganism and/or its spores (that are capable of providing PepO2) is administered to a patient to treat/cure MG. In accordance with one or more such embodiments, the microorganism and spores include, for example and without limitation, Lactococcus lactis cremoris, Lactobacillus helveticus, and Lactobacillus johnsonii and their various strains. An effective amount of the microorganism and/or its spores is an amount that is effective to cause destruction or deactivation of mimics, immunogens and/or antigens that cause or exacerbate MG. In accordance with one or more such embodiments, an effective amount is from about 100 thousand CFU to about 600 billion CFU per dose, where the dose is administered about one or more times per week, or as often as about one to three times daily.

In accordance with one or more embodiments of the present invention, a medicament comprised of an effective amount of parts of, or entire broken up, microorganisms and/or their spores (that are capable of providing PepF) is administered to a patient to treat/cure MG. Parts of the microorganisms and/or spores can be separated and selected, using any one of a number of methods that are well known to those of ordinary skill in the art, for their bioactive properties to help ensure and improve the rate of the destruction or deactivation of mimics, immunogens and/or antigens that cause or exacerbate MG and/or to improve the effectiveness of enzymes in the gastrointestinal or respiratory tract in destroying or deactivating such mimics, immunogens and/or antigens. In accordance with one or more such embodiments, the microorganism and spores include, for example and without limitation, Lactobacillus jensenii, Lactobacillus crispatus, Lactobacillus johnsonii, Lactobacillus plantarum, Lactobacillus helveticus, Lactobacillus amylolyticus, Lactobacillus salivarius, Lactobacillus ultunensis, Lactobacillus rhamnosus, Lactobacillus acidophilus, Lactobacillus delbrueki bulgaricus, Lactobacillus gasseri, Lactobacillus casei, Lactobacillus coleohominis, Lactobacillus fermentum, Lactobacillus paracasei, Lactococcus lactis cremoris, Enterococcus faecalis, Bacillus cereus (spore-forming), and Oenococcus oeni and their various strains. An effective amount of the parts of, or entire broken up, microorganisms and/or their spores is an amount sufficient to cause destruction or deactivation of mimics, immunogens and/or antigens that cause or exacerbate MG. In accordance with one or more such embodiments, an effective amount of parts or entire broken up microorganisms is from about 100 thousand CFU to about 600 billion CFU per dose, where the dose is administered about one or more times per week, or as often as about one to three times daily. Methods for breaking up suitable microorganisms and/or spores are set forth above with respect to MS.

In accordance with one or more embodiments of the present invention, a medicament comprised of an effective amount of parts of, or entire broken up, microorganisms and/or their spores (that are capable of providing PepO2) is administered to a patient to treat/cure MG. Parts of the microorganisms and/or spores can be separated and selected, using any one of a number of methods that are well known to those of ordinary skill in the art, for their bioactive properties to help ensure and improve the rate of the destruction or deactivation of mimics, immunogens or immunogens that cause or exacerbate MG and/or to improve the effectiveness of enzymes in the gastrointestinal or respiratory tract in destroying or deactivating such mimics, immunogens and/or antigens. In accordance with one or more such embodiments, the microorganism and spores include, for example and without limitation, Lactococcus lactis cremoris, Lactobacillus helveticus, and Lactobacillus johnsonii and their various strains. An effective amount of the parts of, or entire broken up, microorganisms and/or their spores is an amount sufficient to cause destruction or deactivation of mimics, immunogens and/or antigens that cause or exacerbate MG. In accordance with one or more such embodiments, an effective amount of parts or entire broken up microorganisms is from about 100 thousand CFU to about 600 billion CFU per dose, where the dose is administered about one or more times per week, or as often as about one to three times daily. Methods for breaking up suitable microorganisms and/or spores are set forth above with respect to MS.

In accordance with one or more embodiments, the above-described medicaments can be administered: orally (the methods relating to oral administration described above with respect to MS may also be applied with respect to MG); rectally (the methods relating to rectal administration and selection of microorganisms as sources of suitable enzymes described above with respect to MS may also be applied with respect to MG); transdermally (the methods relating to transdermal administration described above with respect to MS may also be applied with respect to MG); intravenously (the methods relating to intravenous administration described above with respect to MS may also be applied with respect to MG); and by inhalation (the methods relating to inhalation administration described above with respect to MS may also be applied with respect to MG).

In accordance with one or more embodiments, a medicament comprised of PepF can be combined with other enzymes or probiotics such as, for example and without limitation, subtilisin or Lactobacillus lactis L1A.

Uveoretinitis: In accordance with one or more embodiments of the present invention, a medicament comprised of an effective dose of oligopeptidase F (PepF) is administered to a patient to treat/cure Uveoretinitis. In accordance with one or more such embodiments, an effective dose or amount of PepF is a dose or amount of the protease (for example, in sufficient concentration) that is effective in destroying or deactivating mimics, immunogens antigens that cause or exacerbate Uveoretinitis. The effective amount of endopeptidase F (PepF) administered will depend upon the severity of the disease process (the medicament will be administered one or more, preferably three, doses daily), but will range between about 20 units/day and about 200 units/day where the definition of a unit is an amount of PepF required to cleave one micromole of bradykinin at a pH of 8.0 and a temperature of 40° C.

In accordance with one or more further embodiments, a medicament comprised of an effective dose of endopeptidase O (PepO) is administered to a patient to treat/cure Uveoretinitis. This enzyme specifically hydrolyzes 20-5 amino acids peptides on the N-terminal side of hydrophobic amino acids (see, for example, the following articles: “Enzymatic Ability of Bifidobacterium Animalis Subsp Lactis to Hydrolyze Milk Proteins: Identification and Characterization of Endopeptidase O” by C. Janer, F. Arigoni, B. H. Lee, C, Pelaez and T. Requena in App. Environ. Microbiol., 71 pp. 8460-8465 (2005); “Cloning and expression of an oligopeptidase, PepO, with novel specificity form Lactobacillus rhamnosus HN001” by C. Christensson, H. Bratt, L. J. Collins. T. Coolbear, et al. in App. Environ. Microbiol., 68 pp. 254-262 (2002); “Characterization of an Intracellular Oligopeptidase from Lactobacillus Paracasei” by R. O. Tobiassen, T. Sorhaug and L. Stepaniak in App. Environ. Microbiol., 63 pp. 1284-1287 (1997); “Genetic characterization and physiological role of endopeptidase O from Lactobacillus helveticus CNRZ32.” by Y—S Chen and J. L. Steele in App. Environ. Microbiol., 64 pp. 3411-3415 (1998); “Cloning and Sequencing of the Gene for a Lactococcal Endopeptidase, an Enzyme with Sequence Similarity to Mammalian Enkephalinase” by I. Mierau, P. S. T. Tan, A. J. Haandrikman, J. Kok et al. in J. Bacteriol., 175 pp. 2087-2096 (1993); “Purification and Characterization of an Endopeptidase from Lactococcus Lactis Subsp. Cremoris SK11” by G. C. Pritchard, A. D. Freebairn and T. Coolberg in Microbiol., 140 pp. 923-930 (1994); and “Purification and Characterization of an Endopetidase from Lactococcus Lactis Subsp Cremoris Wg2” by P. S. T. Tan, K. M. Pos and W. N. Koninigs in App. Environ. Microbiol., 57 pp. 3593-3599 (1991)). Endopeptidase O specifically hydrolyzes 20-5 amino acids peptides on the N-terminal side of hydrophobic amino acids, and known immunogens fall in this range. Hence, the PepO will destroy or help destroy mimics, immunogens and/or antigens for CD, and it will also destroy or help destroy small biologically active proline peptides which adversely affect Uveoretinitis patients. Thus, once digested, the smaller peptides can be quickly absorbed and transported into bacterial cells for further destruction to individual amino acids.

In accordance with one or more such embodiments, an effective dose or amount of PepO is a dose or amount of the protease (for example, in sufficient concentration) that is effective in destroying or deactivating mimics, immunogens antigens that cause or exacerbate Uveoretinitis. The effective amount of endopeptidase O (PepO) administered will depend upon the severity of the disease process (the medicament will be administered one or more, preferably three, doses daily), but will range between about 20 units/day and about 200 units/day where the definition of a unit is the amount of PepO required to cleave one micromole of bradykinin at a pH of 6.0 and a temperature of 25° C.

In accordance with one or more embodiments of the present invention, a medicament comprised of an effective amount of a non-pathogenic microorganism and/or its spores (that are capable of providing PepF) is administered to a patient to treat/cure Uveoretinitis. In accordance with one or more such embodiments, the microorganism and spores include, for example and without limitation, Lactobacillus jensenii, Lactobacillus crispatus, Lactobacillus johnsonii, Lactobacillus plantarum, Lactobacillus helveticus, Lactobacillus amylolyticus, Lactobacillus salivarius, Lactobacillus ultunensis, Lactobacillus rhamnosus, Lactobacillus acidophilus, Lactobacillus delbrueki bulgaricus, Lactobacillus gasseri, Lactobacillus casei, Lactobacillus coleohominis, Lactobacillus fermentum, Lactobacillus paracasei, Lactococcus lactis cremoris, Enterococcus faecalis, Bacillus cereus (spore-forming), Campylobacter subtilisin, and Oenococcus oeni and their various strains. An effective amount of the microorganism and/or its spores is an amount that is effective to cause destruction or deactivation of mimics, immunogens and/or antigens that cause or exacerbate Uveoretinitis. In accordance with one or more such embodiments, an effective amount is from about 100 thousand CFU to about 600 billion CFU per dose, where the dose is administered about one or more times per week, or as often as about one to about three times daily.

In accordance with one or more embodiments of the present invention, a medicament comprised of an effective amount of a non-pathenogenic microorganism and/or its spores (that are capable of providing PepO) is administered to a patient to treat/cure Uveoretinitis. Endopeptidase O is found in a large range of bacterial systems. In accordance with one or more such embodiments, the microorganism and spores include, for example and without limitation, Lactobacillus sanfrancisens, Lactobacillus plantarum, Lactobacillus acidophilus, Lactobacillus helveticus, Lactobacillus casei, Lactobacillus reuteri, Lactobacillus salivarius, Lactobacillus gasseri, Lactobacillus rhamnosus, Lactobacillus johnsonii, Lactobacillus jensenii, Lactobacillus amylolyticus, Lactobacillus sakei, Lactobacillus antrii, Lactobacillus paracasei, Lactobacillus ruminis, Lactococcus lactis, Bifidobacterium dentium, Bifidobacterium longum, Bifidobacterium adolescentis, Bifidobacterium animalis, and Oenicoccus oeni and their various strains. An effective amount of the microorganism and/or its spores is an amount that is effective to cause destruction or deactivation of mimics, immunogens and/or antigens that cause or exacerbate Uveoretinitis. In accordance with one or more such embodiments, an effective amount is from about 100 thousand CFU to about 600 billion CFU per dose, where the dose is administered about one or more times per week, or as often as about one to three times daily.

In accordance with one or more embodiments of the present invention, a medicament comprised of an effective amount of parts of, or entire broken up, microorganisms and/or their spores (that are capable of providing PepF) is administered to a patient to treat/cure Uveoretinitis. Parts of the microorganisms and/or spores can be separated and selected, using any one of a number of methods that are well known to those of ordinary skill in the art, for their bioactive properties to help ensure and improve the rate of the destruction or deactivation of mimics, immunogens and/or antigens that cause or exacerbate Uveoretinitis and/or to improve the effectiveness of enzymes in the gastrointestinal or respiratory tract in destroying or deactivating such mimics, immunogens and/or antigens. In accordance with one or more such embodiments, the microorganism and spores include, for example and without limitation, Lactobacillus jensenii, Lactobacillus crispatus, Lactobacillus johnsonii, Lactobacillus plantarum, Lactobacillus helveticus, Lactobacillus amylolyticus, Lactobacillus salivarius, Lactobacillus ultunensis, Lactobacillus rhamnosus, Lactobacillus acidophilus, Lactobacillus delbrueki bulgaricus, Lactobacillus gasseri, Lactobacillus casei, Lactobacillus coleohominis, Lactobacillus fermentum, Lactobacillus paracasei, Lactococcus lactis cremoris, Enterococcus faecalis, Bacillus cereus (spore-forming), Campylobacter subtilisin, and Oenococcus oeni and their various strains. An effective amount of the parts of, or entire broken up, microorganisms and/or their spores is an amount sufficient to cause destruction or deactivation of mimics, immunogens and/or antigens that cause or exacerbate Uveoretinitis. In accordance with one or more such embodiments, an effective amount of parts or entire broken up microorganisms is from about 100 thousand CFU to about 600 billion CFU per dose, where the dose is administered about one or more times per week, or as often as about one to three times daily. Methods for breaking up suitable microorganisms and/or spores are set forth above with respect to MS.

In accordance with one or more embodiments of the present invention, a medicament comprised of an effective amount of parts of, or entire broken up, microorganisms and/or their spores (that are capable of providing PepO) is administered to a patient to treat/cure Uveoretinitis. Parts of the microorganisms and/or spores can be separated and selected, using any one of a number of methods that are well known to those of ordinary skill in the art, for their bioactive properties to help ensure and improve the rate of the destruction or deactivation of mimics, immunogens or immunogens that cause or exacerbate Uveoretinitis and/or to improve the effectiveness of enzymes in the gastrointestinal or respiratory tract in destroying or deactivating such mimics, immunogens and/or antigens. In accordance with one or more such embodiments, the microorganism and spores include, for example and without limitation, Lactobacillus sanfrancisens, Lactobacillus plantarum, Lactobacillus acidophilus, Lactobacillus helveticus, Lactobacillus casei, Lactobacillus reuteri, Lactobacillus salivarius, Lactobacillus gasseri, Lactobacillus rhamnosus, Lactobacillus johnsonii, Lactobacillus jensenii, Lactobacillus amylolyticus, Lactobacillus sakei, Lactobacillus antri, Lactobacillus paracasei, Lactobacillus ruminis, Lactococcus lactis, Bifidobacterium dentium, Bifidobacterium longum, Bifidobacterium adolescentis, Bifidobacterium animalis, and Oenicoccus oeni and their various strains. An effective amount of the parts of, or entire broken up, microorganisms and/or their spores is an amount sufficient to cause destruction or deactivation of mimics, immunogens and/or antigens that cause or exacerbate Uveoretinitis. In accordance with one or more such embodiments, an effective amount of parts or entire broken up microorganisms is from about 100 thousand CFU to about 600 billion CFU per dose, where the dose is administered about one or more times per week, or as often as about one to three times daily. Methods for breaking up suitable microorganisms and/or spores are set forth above with respect to MS.

In accordance with one or more embodiments, the above-described medicaments can be administered: orally (the methods relating to oral administration described above with respect to MS may also be applied with respect to Uveoretinitis); rectally (the methods relating to rectal administration and selection of microorganisms as sources of suitable enzymes described above with respect to MS may also be applied with respect to Uveoretinitis); transdermally (the methods relating to transdermal administration described above with respect to MS may also be applied with respect to Uveoretinitis); intravenously (the methods relating to intravenous administration described above with respect to MS may also be applied with respect to Uveoretinitis); and by inhalation (the methods relating to inhalation administration described above with respect to MS may also be applied with respect to Uveoretinitis).

In accordance with one or more embodiments, a medicament comprised of PepF can be combined with other enzymes or probiotics such as, for example and without limitation, subtilisin or Lactobacillus lactis L1A.

Lupus Erythematosus: In accordance with one or more embodiments of the present invention, a medicament comprised of an effective dose of endopeptidase O2 (PepO2) is administered to a patient to treat/cure Lupus Erythematosis. In accordance with one or more such embodiments, an effective dose or amount of PepO2 is a dose or amount of the PepO2 (for example, in sufficient concentration) that is effective in destroying or deactivating mimics, immunogens antigens that cause or exacerbate Lupus Erythematosus. While endopeptidase O2 retains the ability to break bonds n-terminal to hydrophobic residues, the added specificity for post-proline bonds distinguishes it form other PepO-type endopeptidases (see, for example, an article entitled “Identification and Characterization of Lactobacillus Helveticus PepO2, an Endopeptidase with Post-Proline Specificity” by Y.-S. Chen, J. E. Christensen, J. R. Broadbent and J. L. Steele in Appl. Environ. Microbiol., 69 pp. 1276-1282 (2003)). The effective amount of endopeptidase O2 (PepO2) administered will depend upon the severity of the disease process (the medicament will be administered one or more, preferably three, doses daily), but will range between about 20 units/day and about 200 units/day where the definition of a unit is an amount of PepO2 required to cleave one micromole of BCN (f193-209) at a pH of 6.5 and a temperature of 25° C.

In accordance with one or more further embodiments, a medicament comprised of an effective dose of endonuclease O (PepO) from Bifidobacterium animalis subsp lactis is administered to a patient to treat/cure Lupus Erythematosus. Such PepO will destroy potential lupus erythematosus mimics, immunogens and/or antigens prior to immune activation (see, for example, an article entitled “Enzymatic Ability of Bifidobacterium Animalis Subsp Lactis to Hydrolyze Milk Proteins: Identification and Characterization of Endopeptidase O” by C. Janer, F. Arigoni, B. H. Lee, C, Pelaez and T. Requena in App. Environ. Microbiol., 71 pp. 8460-8465 (2005). While this is a PepO endonuclease, the protease from Bifidobacterium has an ability to hydrolyze pro-pro bonds that has not been reported with other endopeptidase O proteases.

In accordance with one or more such embodiments, an effective dose or amount of PepO is a dose or amount of the protease (for example, in sufficient concentration) that is effective in destroying or deactivating mimics, immunogens antigens that cause or exacerbate Lupus Erythematosus. The effective amount of endopeptidase O (PepO) administered will depend upon the severity of the disease process (the medicament will be administered one or more, preferably three, doses daily), but will range between about 20 units/day and about 200 units/day where the definition of a unit is the amount of PepO from Bifdobacterium animalis subsp. lactis required to cleave one micromole of bradykinin at a pH of 6.0 and a temperature of 25° C.

In accordance with one or more embodiments of the present invention, a medicament comprised of an effective amount of a non-pathenogenic microorganism and/or its spores (that are capable of providing PepO2) is administered to a patient to treat/cure Lupus Erythematosus. In accordance with one or more such embodiments, the microorganism and spores include, for example and without limitation, Lactococcus lactis cremoris, Lactobacillus helveticus, and Lactobacillus johnsonii and their various strains. An effective amount of the microorganism and/or its spores is an amount that is effective to cause destruction or deactivation of mimics, immunogens and/or antigens that cause or exacerbate Lupus Erythematosus. In accordance with one or more such embodiments, an effective amount is from about 100 thousand CFU to about 600 billion CFU per dose, where the dose is administered about one or more times per week, or as often as about one to three times daily.

In accordance with one or more embodiments of the present invention, a medicament comprised of an effective amount of a non-pathenogenic microorganism and/or its spores (that are capable of providing PepO) is administered to a patient to treat/cure Lupus Erythematosus. In accordance with one or more such embodiments, the microorganism and spores include, for example and without limitation, Bifidobacterium animalis subsp lactis and their various strains. An effective amount of the microorganism and/or its spores is an amount that is effective to cause destruction or deactivation of mimics, immunogens and/or antigens that cause or exacerbate Lupus Erythematosus. In accordance with one or more such embodiments, an effective amount is from about 100 thousand CFU to about 600 billion CFU per dose, where the dose is administered about one or more times per week, or as often as about one to three times daily.

In accordance with one or more embodiments of the present invention, a medicament comprised of an effective amount of parts of, or entire broken up, microorganisms and/or their spores (that are capable of providing PepO2) is administered to a patient to treat/cure Lupus Erythematosus. Parts of the microorganisms and/or spores can be separated and selected, using any one of a number of methods that are well known to those of ordinary skill in the art, for their bioactive properties to help ensure and improve the rate of the destruction or deactivation of mimics, immunogens or immunogens that cause or exacerbate Lupus Erythematosus and/or to improve the effectiveness of enzymes in the gastrointestinal or respiratory tract in destroying or deactivating such mimics, immunogens and/or antigens. In accordance with one or more such embodiments, the microorganism and spores include, for example and without limitation, Lactococcus lactis cremoris, Lactobacillus helveticus, and Lactobacillus johnsonii and their various strains. An effective amount of the parts of, or entire broken up, microorganisms and/or their spores is an amount sufficient to cause destruction or deactivation of mimics, immunogens and/or antigens that cause or exacerbate Lupus Erythematosus. In accordance with one or more such embodiments, an effective amount of parts or entire broken up microorganisms is from about 100 thousand CFU to about 600 billion CFU per dose, where the dose is administered about one or more times per week, or as often as about one to three times daily. Methods for breaking up suitable microorganisms and/or spores are set forth above with respect to MS.

In accordance with one or more embodiments of the present invention, a medicament comprised of an effective amount of parts of, or entire broken up, microorganisms and/or their spores (that are capable of providing PepO) is administered to a patient to treat/cure Lupus Erythematosus. Parts of the microorganisms and/or spores can be separated and selected, using any one of a number of methods that are well known to those of ordinary skill in the art, for their bioactive properties to help ensure and improve the rate of the destruction or deactivation of mimics, immunogens and/or antigens that cause or exacerbate Lupus Erythematosus and/or to improve the effectiveness of enzymes in the gastrointestinal or respiratory tract in destroying or deactivating such mimics, immunogens and/or antigens. In accordance with one or more such embodiments, the microorganism and spores include, for example and without limitation, Bifidobacterium animalis subsp lactis and their various strains. An effective amount of the parts of, or entire broken up, microorganisms and/or their spores is an amount sufficient to cause destruction or deactivation of mimics, immunogens and/or antigens that cause or exacerbate Lupus Erythematosus. In accordance with one or more such embodiments, an effective amount of parts or entire broken up microorganisms is from about 100 thousand CFU to about 600 billion CFU per dose, where the dose is administered about one or more times per week, or as often as about one to three times daily. Methods for breaking up suitable microorganisms and/or spores are set forth above with respect to MS.

In accordance with one or more embodiments, the above-described medicaments can be administered: orally (the methods relating to oral administration described above with respect to MS may also be applied with respect to Lupus Erythematosus); rectally (the methods relating to rectal administration and selection of microorganisms as sources of suitable enzymes described above with respect to MS may also be applied with respect to Lupus Erythematosus); transdermally (the methods relating to transdermal administration described above with respect to MS may also be applied with respect to Lupus Erythematosus); intravenously (the methods relating to intravenous administration described above with respect to MS may also be applied with respect to Lupus Erythematosus); and by inhalation (the methods relating to inhalation administration described above with respect to MS may also be applied with respect to Lupus Erythematosus).

In accordance with one or more embodiments, a medicament comprised of PepO or PepO2 can be combined with other enzymes or probiotics such as, for example and without limitation, subtilisin or Lactobacillus lactis L1A.

Ulcerative Colitis: In accordance with one or more embodiments of the present invention, a medicament comprised of an effective dose of oligopeptidase F (PepF) is administered to a patient to treat/cure Ulcerative Colitis. In accordance with one or more such embodiments, an effective dose or amount of PepF is a dose or amount of the protease (for example, in sufficient concentration) that is effective in destroying or deactivating mimics, immunogens antigens that cause or exacerbate Ulcerative Colitis. The effective amount of endopeptidase F (PepF) administered will depend upon the severity of the disease process (the medicament will be administered one or more, preferably three, doses daily), but will range between about 20 units/day and about 200 units/day where the definition of a unit is an amount of PepF required to cleave one micromole of bradykinin at a pH of 8.0 and a temperature of 40° C.

In accordance with one or more further embodiments, a medicament comprised of an effective dose of endopeptidase O (PepO) is administered to a patient to treat/cure Ulcerative Colitis. Endopeptidase O is found in a large range of bacterial systems, and specifically hydrolyzes 20-5 amino acids peptides on the N-terminal side of hydrophobic amino acids. Hence, PepO will destroy or help destroy mimics, immunogens and/or antigens for Ulcerative Colitis so that they can be absorbed and transported in bacterial cells for further destruction to individual amino acids. This process will reduce the concentration of small proline containing peptides, especially C-terminal proline peptides which can contribute to hormonal aberrations. Known immunogens fall in this range. Once digested, the smaller peptides can be quickly absorbed and transported in the bacterial cells for further destruction to individual amino acids (see, for example, the following articles: “Linear Epitope Mapping of a SmB/B′ Polypeptide” by J. A. James and J. B. Harley in Immunol., 148 pp. 2074-9 (1992); “Cloning and expression of an oligopeptidase, PepO, with novel specificity form Lactobacillus rhamnosus HN001” by C. Christensson, H. Bratt, L. J. Collins. T. Coolbear, et al. in App. Environ. Microbiol., 68 pp. 254-262 (2002); “Characterization of an Intracellular Oligopeptidase from Lactobacillus Paracasei” by R. O. Tobiassen, T. Sorhaug and L. Stepaniak in App. Environ. Microbiol., 63 pp. 1284-1287 (1997); “Genetic characterization and physiological role of endopeptidase O from Lactobacillus helveticus CNRz32.” by Y—S Chen and J. L. Steele in App. Environ. Microbiol., 64 pp. 3411-3415 (1998); “Cloning and Sequencing of the Gene for a Lactococcal Endopeptidase, an Enzyme with Sequence Similarity to Mammalian Enkephaliinase” by I. Mierau, P. S. T. Tan, A. J. Haandrikman, J. Kok et al. in J. Bacteriol., 175 pp. 2087-2096 (1993); “Purification and Characterization of an Endopeptidase from Lactococcus Lactis Subsp. Cremoris SK11” by G. C. Pritchard, A. D. Freebairn and T. Coolberg in Microbiol., 140 pp. 923-930 (1994); and “Purification and Characterization of an Endopetidase from Lactococcus Lactis Subsp Cremoris Wg2” by P. S. T. Tan, K. M. Pos and W. N. Koninigs in App. Environ. Microbiol., 57 pp. 3593-3599 (1991)).

In accordance with one or more such embodiments, an effective dose or amount of PepO is a dose or amount of the protease (for example, in sufficient concentration) that is effective in destroying or deactivating mimics, immunogens antigens that cause or exacerbate Ulcerative Colitis. The effective amount of endopeptidase O (PepO) administered will depend upon the severity of the disease process (the medicament will be administered one or more, preferably three, doses daily), but will range between about 20 units/day and about 200 units/day where the definition of a unit is the amount of PepO required to cleave one micromole of bradykinin at a pH of 6.0 and a temperature of 25° C.

In accordance with one or more embodiments of the present invention, a medicament comprised of an effective amount of a non-pathogenic microorganism and/or its spores (that are capable of providing PepF) is administered to a patient to treat/cure Ulcerative Colitis. In accordance with one or more such embodiments, the microorganism and spores include, for example and without limitation, Lactobacillus jensenii, Lactobacillus crispatus, Lactobacillus johnsonii, Lactobacillus plantarum, Lactobacillus helveticus, Lactobacillus amylolyticus, Lactobacillus salivarius, Lactobacillus ultunensis, Lactobacillus rhamnosus, Lactobacillus acidophilus, Lactobacillus delbrueki bulgaricus, Lactobacillus gasseri, Lactobacillus casei, Lactobacillus coleohominis, Lactobacillus fermentum, Lactobacillus paracasei, Lactococcus lactis cremoris, Enterococcus faecalis, Bacillus cereus (spore-forming), and Oenococcus oeni and their various strains. An effective amount of the microorganism and/or its spores is an amount that is effective to cause destruction or deactivation of mimics, immunogens and/or antigens that cause or exacerbate Ulcerative Colitis. In accordance with one or more such embodiments, an effective amount is from about 100 thousand CFU to about 600 billion CFU per dose, where the dose is administered about one or more times per week, or as often as about one to about three times daily.

In accordance with one or more embodiments of the present invention, a medicament comprised of an effective amount of a non-pathenogenic microorganism and/or its spores (that are capable of providing PepO) is administered to a patient to treat/cure Ulcerative Colitis. In accordance with one or more such embodiments, the microorganism and spores include, for example and without limitation, Lactobacillus san francisens, Lactobacillus plantarum, Lactobacillus acidophilus, Lactobacillus helveticus, Lactobacillus casei, Lactobacillus reuteri, Lactobacillus salivarius, Lactobacillus gasseri, Lactobacillus rhamnosus, Lactobacillus johnsonii, Lactobacillus jensenii, Lactobacillus amylolyticus, Lactobacillus sakei, Lactobacillus antri, Lactobacillus paracasei, Lactobacillus ruminis, Lactococcus lactis, Bifidobacterium dentium, Bifidobacterium longum, Bifidobacterium adolescentis, Bifidobacterium animalis, and Oenicoccus oeni and their various strains. An effective amount of the microorganism and/or its spores is an amount that is effective to cause destruction or deactivation of mimics, immunogens and/or antigens that cause or exacerbate Ulcerative Colitis. In accordance with one or more such embodiments, an effective amount is from about 100 thousand CFU to about 600 billion CFU per dose, where the dose is administered about one or more times per week, or as often as about one to three times daily.

In accordance with one or more embodiments of the present invention, a medicament comprised of an effective amount of parts of, or entire broken up, microorganisms and/or their spores (that are capable of providing PepF) is administered to a patient to treat/cure Ulcerative Colitis. Parts of the microorganisms and/or spores can be separated and selected, using any one of a number of methods that are well known to those of ordinary skill in the art, for their bioactive properties to help ensure and improve the rate of the destruction or deactivation of mimics, immunogens and/or antigens that cause or exacerbate Ulcerative Colitis and/or to improve the effectiveness of enzymes in the gastrointestinal or respiratory tract in destroying or deactivating such mimics, immunogens and/or antigens. In accordance with one or more such embodiments, the microorganism and spores include, for example and without limitation, Lactobacillus jensenii, Lactobacillus crispatus, Lactobacillus johnsonii, Lactobacillus plantarum, Lactobacillus helveticus, Lactobacillus amylolyticus, Lactobacillus salivarius, Lactobacillus ultunensis, Lactobacillus rhamnosus, Lactobacillus acidophilus, Lactobacillus delbrueki bulgaricus, Lactobacillus gasseri, Lactobacillus casei, Lactobacillus coleohominis, Lactobacillus fermentum, Lactobacillus paracasei, Lactococcus lactis cremoris, Enterococcus faecalis, Bacillus cereus (spore-forming), and Oenococcus oeni and their various strains. An effective amount of the parts of, or entire broken up, microorganisms and/or their spores is an amount sufficient to cause destruction or deactivation of mimics, immunogens and/or antigens that cause or exacerbate Ulcerative Colitis. In accordance with one or more such embodiments, an effective amount of parts or entire broken up microorganisms is from about 100 thousand CFU to about 600 billion CFU per dose, where the dose is administered about one or more times per week, or as often as about one to three times daily. Methods for breaking up suitable microorganisms and/or spores are set forth above with respect to MS.

In accordance with one or more embodiments of the present invention, a medicament comprised of an effective amount of parts of, or entire broken up, microorganisms and/or their spores (that are capable of providing PepO) is administered to a patient to treat/cure Ulcerative Colitis. Parts of the microorganisms and/or spores can be separated and selected, using any one of a number of methods that are well known to those of ordinary skill in the art, for their bioactive properties to help ensure and improve the rate of the destruction or deactivation of mimics, immunogens or immunogens that cause or exacerbate Ulcerative Colitis and/or to improve the effectiveness of enzymes in the gastrointestinal or respiratory tract in destroying or deactivating such mimics, immunogens and/or antigens. In accordance with one or more such embodiments, the microorganism and spores include, for example and without limitation, Lactobacillus sanfrancisens, Lactobacillus plantarum, Lactobacillus acidophilus, Lactobacillus helveticus, Lactobacillus casei, Lactobacillus reuteri, Lactobacillus salivarius, Lactobacillus gasseri, Lactobacillus rhamnosus, Lactobacillus johnsonii, Lactobacillus jensenii, Lactobacillus amylolyticus, Lactobacillus sakei, Lactobacillus antri, Lactobacillus paracasei, Lactobacillus ruminis, Lactococcus lactis, Bifidobacterium dentium, Bifidobacterium longum, Bifidobacterium adolescentis, Bifidobacterium animalis, and Oenicoccus oeni and their various strains. An effective amount of the parts of, or entire broken up, microorganisms and/or their spores is an amount sufficient to cause destruction or deactivation of mimics, immunogens and/or antigens that cause or exacerbate Ulcerative Colitis. In accordance with one or more such embodiments, an effective amount of parts or entire broken up microorganisms is from about 100 thousand CFU to about 600 billion CFU per dose, where the dose is administered about one or more times per week, or as often as about one to three times daily. Methods for breaking up suitable microorganisms and/or spores are set forth above with respect to MS.

In accordance with one or more embodiments, the above-described medicaments can be administered: orally (the methods relating to oral administration described above with respect to MS may also be applied with respect to Ulcerative Colitis); rectally (the methods relating to rectal administration and selection of microorganisms as sources of suitable enzymes described above with respect to MS may also be applied with respect to Ulcerative Colitis); transdermally (the methods relating to transdermal administration described above with respect to MS may also be applied with respect to Ulcerative Colitis); intravenously (the methods relating to intravenous administration described above with respect to MS may also be applied with respect to Ulcerative Colitis); and by inhalation (the methods relating to inhalation administration described above with respect to MS may also be applied with respect to Ulcerative Colitis).

In accordance with one or more embodiments, a medicament comprised of PepF can be combined with other enzymes or probiotics such as, for example and without limitation, subtilisin or Lactobacillus lactis L1A.

Celiac Disease (CD): In accordance with one or more embodiments of the present invention, a medicament comprised of an effective dose or amount of Oenococcus oeni is administered to a patient to treat/cure CD. Oenicoccus oeni is a bacterial agent used as a starter inoculum in wine making (see, for example, the following articles: “Saccharomyces Cerevisiae-Oenococcus Oeni Interactions in Wine: Current Knowledge and Perspectives” by H. Alexandre, P. J. Costello, F. Rremize, J. Guzzo et al., in Int. J. Food Microbiol., 93 pp. 141-54 (2004); “A new approach for selection of Oenococcus oeni strains in order to produce malolactic starters” by F. Coucheney, N. Desroche, M. Bou, R. Tourdot-Marechal, L. Dulau and J. Guzzo in Int. J. Food Microbiol., 105 pp. 463-70 (2005); and “Characterization and Technological Properties of Oenococcus Oeni Strains from Wine Spontaneous Malolactic Fermentations: a Framework for Selection of New Starter Cultures” by L. Solieri, F. Genova, M. De Paola and P. Giudici in J. Appl. Microbiol., 108 pp. 285-98 (2010)). An effective dose or amount of Oenococcus oeni is a dose or amount of Oenococcus oeni that is effective in destroying or deactivating mimics, immunogens and/or antigens that cause or exacerbate CD. The effective amount of Oenococcus oeni administered will depend upon the severity of the disease process, but each dose should contain from about 1×10⁵ to about 600×10⁹ CFU per dose of each strain, and preferably >1×10⁷ CFU per dose of each single strain. It is believed that Oenococcus oeni provides a series of proline digesting enzymes which destroy small proline, potentially harmful, peptides (see, for example, the following articles: “Purification and Partial Characterization of Oenococcus Oeni Exoprotease” by M. E. Farias and M. C. Manca de Nadra in FEBS Lett., 185 pp. 263-6 (2000); “Characterization of EprA, a Major Extracellular Protein of Oenococcus Oeni with Protease Activity” by P. Folio, J. F. Ritt, H. Alexander and F. Remize in Int. J. Food Microbiol., 127 pp. 26-31 (2008); and “Peptidases Specific for Proline-Containing Peptides and Their Unusual Peptide-Dependent Regulation in Oenococcus oeni” by J. F. Ritt, F. Remize, C. Grandvalet, J. Guzzo, D. Atlan and H. Alexandre in J. Appl. Microbiol., 106 pp. 801-13 (2009)). Further, Oenococcus Oeni has an efficient transport system for 2-5 amino acid oligopeptides (see, for example, the following articles: “Oligopeptide Assimilation and Transport by Oenococcus Oeni” by J. F. Ritt, M. Guilloux-Benatier, J. Guzzo, H. Alexandre and F. Remize in J. Appl. Microbiol., 104 pp. 573-80 (2008) and “Oenococcus Oeni Preference for Peptides: Qualitative and Quantitative Analysis of Nitrogen Assimilation” by F. Remize, A. Gaudin, Y. Kong, J. Guzzo et al. in Arch. Microbiol., 185 pp. 459-69 (2006)). It is believed that this treatment will help reduce the concentration of small hormonally active proteins produced from gluten digestion from contributing negatively biochemically, and will address a problem (see, for example, an article entitled “The Effects of ALV003 Predigestion of Gluten on Immune Response and Symptoms in Celiac Disease in Vivo” by J. A. Tye-Din, R. P. Anderson, R. A. French, G. J Brown et al. in Clin. Immunol., 134 pp. 289-295 (2010)) of clinical signs persisting and exacerbating after gluten immunogens have been destroyed.

There are many procedures that are well known to those of ordinary skill in the art for preparing Oenococcus oeni. The following lists some of the more efficient methods: (a) Oenococcus oeni strains can be cultured at 25° C. in a medium adjusted to pH 5 and containing the following (per liter) (i) yeast extract, 4 g; (ii) beef extract, 8 g; (iii) Bacto Peptone, 10 g; (iv) glucose, 10 g; (v) fructose, 10 g; (vi) malic acid, 10 g; (vii) KH₂PO₄, 2 g; (viii) MgSO₄-7H₂O, 0.2 g; (ix) MnSO₄—H₂O, 0.1 g; and (x) Tween 80, 1 ml.; (b) strains can be cultured at 30° C. in FT80 medium (see, for example, the following articles: “Membrane Fluidity Adjustments in Ethanol Stressed Oenococcus Oeni cells” by M. Gracis da Silveira, E. A. Golovina, E. Hoekstra, F. M. Rombouts and T Abee in Appl. Environ. Microbiol., 69 pp. 5826-5832 (2003); “Medium for screening Leuconostic Oenos Strains Defective in Malolactic Fermentation” by J. Cavin, J. Prevost, J. Lin, P. Schmitt and C. Davies in Appl. Environ. Microbiol., 55 pp. 751-753 (1989)) at pH 4.5 without the addition of Tween but containing 10 g of DL-malic acid per liter where: (i) the glucose and fructose should be autoclaved separately and added to the medium just before inoculation at final concentrations of 2 and 8 g/liter, and (ii) stock cultures (kept frozen at −80° C.) are to be grown until early stationary phase (48 hr), diluted 100 fold in fresh medium, and incubated for 24 hrs.; and (c) Oenococcus oeni strains can be cultured at 30 C and pH 5.3 in FT80 medium modified as described by an article entitled “Lactic Acid Bacteria in the Quality Improvement and Depreciation of Wine” by A. Louvaud-Funel in Antonie Leeuwenhoek, 76 pp. 317-331 (1999)).

In accordance with one or more further embodiments, a medicament comprised of an effective dose of endopeptidase O (PepO) or a combination of PepO and Oneoccocus oeni will be administered to a patient to treat/cure CD. Endopeptidase O specifically hydrolyzes 20-5 amino acids peptides on the N-terminal side of hydrophobic amino acids, and known immunogens fall in this range. Hence, the PepO will destroy or help destroy mimics, immunogens and/or antigens for CD, and it will also destroy or help destroy small biologically active proline peptides which adversely effect CD patients. Thus, once digested, smaller peptides can be quickly absorbed and transported into bacterial cells for further destruction to individual amino acids. As a result, the concentration of small proline containing peptides, especially C-terminal proline peptides that can contribute to hormonal adversities, are reduced.

In accordance with one or more such embodiments, an effective dose or amount of PepO or a combination of PepO and Oneococcus oeni is a dose or amount (for example, in sufficient concentration) that is effective in destroying or deactivating mimics, immunogens antigens that cause or exacerbate CD. The effective amount of PepO or a combination of PepO and Oneococcus oeni administered will depend upon the severity of the disease process (the medicament will be administered one or more, preferably three, doses daily), but the amount of the PepO in the dose will range between about 20 units/day and about 200 units/day where the definition of a unit is the amount of PepO required to cleave one micromole of bradykinin at a pH of 6.0 and a temperature of 25° C. and the amount of the Oneococcus oeni in a dose will range from about 1×10⁵ to about 600×10¹¹ CFU per dose of each strain, and preferably >1×10⁷ CFU per dose of each single strain.

In accordance with one or more further embodiments, a medicament comprised of an effective dose of endopeptidase O2 (PepO2) or a combination of PepO2 and Oneococcus oeni is administered to a patient to treat/cure CD. The Lactobacillus helveticus PepO2 enzyme contains the zinc dependent metalloprotease motif HEXXH, and exhibits levels of amino acid homology of 72, 61, 59 and 53% to L. helveticus PepO, Lactococcus lactis PepO2, Lactococcus lactis PepO and Lactobacillus rhamnosus PepO, respectively. While it retains the specificity for bonds n-terminal to hydrophobic amino acids, its added specificity for post-proline bonds distinguishes it from other PepO-type endopeptidases (see, for example, an article entitled “Identification and Characterization of Lactobacillus Helveticus PepO2, an Endopeptidase with Post-Proline Specificity” by Y.-S. Chen, J. E. Christensen, J. R. Broadbent and J. L. Steele in Appl. Environ. Microbiol., 69 pp. 1276-1282 (2003)).

In accordance with one or more such embodiments, an effective dose or amount of endopeptidase O2 (PepO2) or a combination of PepO2 and Oneococcus oeni is a dose or amount (for example, in sufficient concentration) that is effective in destroying or deactivating mimics, immunogens antigens that cause or exacerbate CD. The effective amount of endopeptidase O2 (PepO2) or a combination of PepO2 and Oneococcus oeni administered will depend upon the severity of the disease process (the medicament will be administered one or more, preferably three, doses daily), and the amount of the PepO2 in the dose will range between about 20 units/day and about 200 units/day where the definition of a unit is the amount of PepO2 required to cleave one micromole of BCN (f193-209) at a pH of 6.5 and a temperature of 25° C. and the amount of the Oneococcus oeni in the dose will range from about 1×10⁵ to about 600×10⁹ CFU per dose of each strain, and preferably >1×10⁷ CFU per dose of each single strain.

In accordance with one or more further embodiments, a medicament comprised of an effective dose of endopeptidase F (PepF) or a combination of PepF and Oneococcus oeni is administered to a patient to treat/cure CD. PepF, also a metallopeptidase, hydrolyzes 21-5 amino acid peptides on the N-terminal side of hydrophobic amino acids. Its activity is similar to PepO, but it is less active toward proline bonds.

In accordance with one or more such embodiments, an effective dose or amount of endopeptidase F (PepF) or a combination of PepF and Oneococcus oeni is a dose or amount (for example, in sufficient concentration) that is effective in destroying or deactivating mimics, immunogens antigens that cause or exacerbate CD. The effective amount of an effective dose or amount of PepF or a combination of PepF and Oneococcus oeni administered will depend upon the severity of the disease process (the medicament will be administered one or more, preferably three, doses daily), and the amount of the PepF in the dose will range between about 20 units/day and about 200 units/day where the definition of a unit is the amount of PepF required to cleave one micromole of bradykinin at a pH of 8.0 and a temperature of 40° C. and the amount of the Oneococcus oeni in the dose will range from about 1×10⁵ to about 600×10⁹ CFU per dose of each strain, and preferably >1×10⁷ CFU per dose of each single strain.

In accordance with one or more further embodiments of the present invention, a medicament comprised of an effective amount of a non-pathenogenic microorganism and/or its spores (that are capable of providing PepO) or a combination of Oneococcus oeni and a non-pathenogenic microorganism and/or its spores (that are capable of providing PepO) is administered to a patient to treat/cure CD. In accordance with one or more such embodiments, the microorganism and spores include, for example and without limitation, Lactobacillus sanfrancisens, Lactobacillus plantarum, Lactobacillus acidophilus, Lactobacillus helveticus, Lactobacillus casei, Lactobacillus reuteri, Lactobacillus salivarius, Lactobacillus gasseri, Lactobacillus rhamnosus, Lactobacillus johnsonii, Lactobacillus jensenii, Lactobacillus amylolyticus, Lactobacillus sakei, Lactobacillus antri, Lactobacillus paracasei, Lactobacillus ruminis, Lactococcus lactis, Bifidobacterium dentium, Bifidobacterium longum, Bifidobacterium adolescentis, Bifidobacterium animalis, and Oenicoccus oeni and their various strains. In accordance with one or more such embodiments, an effective dose or amount of the non-pathenogenic microorganism and/or its spores (that are capable of providing PepO) or a combination of Oneococcus oeni and a non-pathenogenic microorganism and/or its spores (that are capable of providing PepO) is an amount that is effective to cause destruction or deactivation of mimics, immunogens and/or antigens that cause or exacerbate Celiac Disease. The effective dose administered will depend upon the severity of the disease process (the medicament will be administered one or more, preferably three, doses daily), but the amount of the microorganism and spores in the dose will range from about 100 thousand CFU to about 600 billion CFU per dose, and the amount of the Oneococcus oeni in a dose will range from about 1×10⁵ to about 600×10⁹ CFU per dose of each strain, and preferably >1×10⁷ CFU per dose of each single strain.

In accordance with one or more further embodiments of the present invention, a medicament comprised of an effective amount of a non-pathenogenic microorganism and/or its spores (that are capable of providing PepO2) or a combination of Oenococcus oeni and a non-pathenogenic microorganism and/or its spores (that are capable of providing PepO2) is administered to a patient to treat/cure CD. In accordance with one or more such embodiments, the microorganism and spores include, for example and without limitation, Lactococcus lactis cremoris, Lactobacillus helveticus, and Lactobacillus johnsonii and their various strains. In accordance with one or more such embodiments, an effective dose or amount of the non-pathenogenic microorganism and/or its spores (that are capable of providing PepO2) or a combination of Oenococcus oeni and a non-pathenogenic microorganism and/or its spores (that are capable of providing PepO2) is an amount that is effective to cause destruction or deactivation of mimics, immunogens and/or antigens that cause or exacerbate Celiac Disease. The effective dose administered will depend upon the severity of the disease process (the medicament will be administered one or more, preferably three, doses daily), but the amount of the microorganism and spores in the dose will range from about 100 thousand CFU to about 600 billion CFU per dose, and the amount of the Oenococcus oeni in a dose will range from about 1×10⁵ to about 600×10⁹ CFU per dose of each strain, and preferably >1×10⁷ CFU per dose of each single strain.

In accordance with one or more further embodiments of the present invention, a medicament comprised of an effective amount of a non-pathenogenic microorganism and/or its spores (that are capable of providing PepF) or a combination of Oenococcus oeni and a non-pathenogenic microorganism and/or its spores (that are capable of providing PepF) is administered to a patient to treat/cure CD. In accordance with one or more such embodiments, the microorganism and spores include, for example and without limitation, but not limited to, Lactobacillus jensenii, Lactobacillus crispatus, Lactobacillus johnsonii, Lactobacillus plantarum, Lactobacillus helveticus, Lactobacillus amylolyticus, Lactobacillus salivarius, Lactobacillus ultunensis, Lactobacillus rhamnosus, Lactobacillus acidophilus, Lactobacillus delbrueki bulgaricus, Lactobacillus gasseri, Lactobacillus casei, Lactobacillus coleohominis, Lactobacillus fermentum, Lactobacillus paracasei, Lactococcus lactis cremoris, Enterococcus faecalis, Bacillus cereus (spore-forming), Campylobacter subtilisin, and Oenococcus oeni and their various strains. In accordance with one or more such embodiments, an effective dose or amount of the non-pathenogenic microorganism and/or its spores (that are capable of providing PepF) or a combination of Oenococcus oeni and a non-pathenogenic microorganism and/or its spores (that are capable of providing PepF) is an amount that is effective to cause destruction or deactivation of mimics, immunogens and/or antigens that cause or exacerbate Celiac Disease. The effective dose administered will depend upon the severity of the disease process (the medicament will be administered one or more, preferably three, doses daily), but the amount of the microorganism and spores in the dose will range from about 100 thousand CFU to about 600 billion CFU per dose, and the amount of the Oenococcus oeni in a dose will range from about 1×10⁵ to about 600×10⁹ CFU per dose of each strain, and preferably >1×10⁷ CFU per dose of each single strain.

In accordance with one or more further embodiments of the present invention, a medicament comprised of an effective amount of parts of, or entire broken up, microorganisms and/or their spores (that are capable of providing PepO) or a combination of Oenococcus oeni and parts of, or entire broken up, microorganisms and/or their spores (that are capable of providing PepO) is administered to a patient to treat/cure CD. Parts of the microorganisms and/or spores can be separated and selected, using any one of a number of methods that are well known to those of ordinary skill in the art, for their bioactive properties to help ensure and improve the rate of the destruction or deactivation of mimics, immunogens and/or antigens that cause or exacerbate CD and/or to improve the effectiveness of enzymes in the gastrointestinal or respiratory tract in destroying or deactivating such mimics, immunogens and/or antigens. In accordance with one or more such embodiments, the microorganism and spores include, for example and without limitation, Lactobacillus sanfrancisens, Lactobacillus plantarum, Lactobacillus acidophilus, Lactobacillus helveticus, Lactobacillus casei, Lactobacillus reuteri, Lactobacillus salivarius, Lactobacillus gasseri, Lactobacillus rhamnosus, Lactobacillus johnsonii, Lactobacillus jensenii, Lactobacillus amylolyticus, Lactobacillus sakei, Lactobacillus antri, Lactobacillus paracasei, Lactobacillus ruminis, Lactococcus lactis, Bifidobacterium dentium, Bifidobacterium longum, Bifidobacterium adolescentis, Bifidobacterium animalis, and Oenicoccus oeni and their various strains. In accordance with one or more such embodiments, an effective dose or amount of parts of, or entire broken up, microorganisms and/or their spores (that are capable of providing PepO) or a combination of Oenococcus oeni and parts of, or entire broken up, microorganisms and/or their spores (that are capable of providing PepO) is an amount that is effective to cause destruction or deactivation of mimics, immunogens and/or antigens that cause or exacerbate Celiac Disease. The effective dose administered will depend upon the severity of the disease process (the medicament will be administered one or more, preferably three, doses daily), but the amount of parts of, or entire broken up, microorganisms and/or their spores will range from about 100 thousand CFU to about 600 billion CFU per dose, and the amount of the Oenococcus oeni in a dose will range from about 1×10⁵ to about 600×10⁹ CFU per dose of each strain, and preferably >1×10⁷ CFU per dose of each single strain. Methods for breaking up suitable microorganisms and/or spores are set forth above with respect to MS.

In accordance with one or more further embodiments of the present invention, a medicament comprised of an effective amount of parts of, or entire broken up, microorganisms and/or their spores (that are capable of providing PepO2) or a combination of Oenococcus oeni and parts of, or entire broken up, microorganisms and/or their spores (that are capable of providing PepO2) is administered to a patient to treat/cure CD. Parts of the microorganisms and/or spores can be separated and selected, using any one of a number of methods that are well known to those of ordinary skill in the art, for their bioactive properties to help ensure and improve the rate of the destruction or deactivation of mimics, immunogens and/or antigens that cause or exacerbate CD and/or to improve the effectiveness of enzymes in the gastrointestinal or respiratory tract in destroying or deactivating such mimics, immunogens and/or antigens. In accordance with one or more such embodiments, the microorganism and spores include, for example and without limitation, Lactococcus lactis cremoris, Lactobacillus helveticus, and Lactobacillus johnsonii and their various strains. In accordance with one or more such embodiments, an effective dose or amount of parts of, or entire broken up, microorganisms and/or their spores (that are capable of providing PepO2) or a combination of Oenococcus oeni and parts of, or entire broken up, microorganisms and/or their spores (that are capable of providing PepO2) is an amount that is effective to cause destruction or deactivation of mimics, immunogens and/or antigens that cause or exacerbate Celiac Disease. The effective dose administered will depend upon the severity of the disease process (the medicament will be administered one or more, preferably three, doses daily), but the amount of parts of, or entire broken up, microorganisms and/or their spores will range from about 100 thousand CFU to about 600 billion CFU per dose, and the amount of the Oenococcus oeni in a dose will range from about 1×10⁵ to about 600×10⁹ CFU per dose of each strain, and preferably >1×10⁷ CFU per dose of each single strain. Methods for breaking up suitable microorganisms and/or spores are set forth above with respect to MS.

In accordance with one or more further embodiments of the present invention, a medicament comprised of an effective amount of parts of, or entire broken up, microorganisms and/or their spores (that are capable of providing PepF) or a combination of Oenococcus oeni and parts of, or entire broken up, microorganisms and/or their spores (that are capable of providing PepF) is administered to a patient to treat/cure CD. Parts of the microorganisms and/or spores can be separated and selected, using any one of a number of methods that are well known to those of ordinary skill in the art, for their bioactive properties to help ensure and improve the rate of the destruction or deactivation of mimics, immunogens and/or antigens that cause or exacerbate CD and/or to improve the effectiveness of enzymes in the gastrointestinal or respiratory tract in destroying or deactivating such mimics, immunogens and/or antigens. In accordance with one or more such embodiments, the microorganism and spores include, for example and without limitation, Lactobacillus jensenii, Lactobacillus crispatus, Lactobacillus johnsonii, Lactobacillus plantarum, Lactobacillus helveticus, Lactobacillus amylolyticus, Lactobacillus salivarius, Lactobacillus ultunensis, Lactobacillus rhamnosus, Lactobacillus acidophilus, Lactobacillus delbrueki bulgaricus, Lactobacillus gasseri, Lactobacillus casei, Lactobacillus coleohominis, Lactobacillus fermentum, Lactobacillus paracasei, Lactococcus lactis cremoris, Enterococcus faecalis, Bacillus cereus (spore-forming), and Oenococcus oeni and their various strains. In accordance with one or more such embodiments, an effective dose or amount of parts of, or entire broken up, microorganisms and/or their spores (that are capable of providing PepF) or a combination of Oenococcus oeni and parts of, or entire broken up, microorganisms and/or their spores (that are capable of providing PepF) is an amount that is effective to cause destruction or deactivation of mimics, immunogens and/or antigens that cause or exacerbate Celiac Disease. The effective dose administered will depend upon the severity of the disease process (the medicament will be administered one or more, preferably three, doses daily), but the amount of parts of, or entire broken up, microorganisms and/or their spores will range from about 100 thousand CFU to about 600 billion CFU per dose, and the amount of the Oenococcus oeni in a dose will range from about 1×10⁵ to about 600×10⁹ CFU per dose of each strain, and preferably >1×10⁷ CFU per dose of each single strain. Methods for breaking up suitable microorganisms and/or spores are set forth above with respect to MS.

In accordance with one or more embodiments, the above-described medicaments can be administered: orally (the methods relating to oral administration described above with respect to MS may also be applied with respect to CD); rectally (the methods relating to rectal administration and selection of microorganisms as sources of suitable enzymes described above with respect to MS may also be applied with respect to CD); transdermally (the methods relating to transdermal administration described above with respect to MS may also be applied with respect to CD); intravenously (the methods relating to intravenous administration described above with respect to MS may also be applied with respect to CD); and by inhalation (the methods relating to inhalation administration described above with respect to MS may also be applied with respect to CD).

Anorexia Nervosa: In accordance with one or more embodiments of the present invention, a medicament comprised of an effective dose of oligopeptidase F (PepF) is administered to a patient to treat/cure Anorexia Nervosa. In accordance with one or more such embodiments, an effective dose or amount of PepF is a dose or amount of the protease (for example, in sufficient concentration) that is effective in destroying or deactivating mimics, immunogens antigens that cause or exacerbate Anorexia Nervosa. The effective amount of endopeptidase F (PepF) administered will depend upon the severity of the disease process (the medicament will be administered one or more, preferably three, doses daily), but will range between about 20 units/day and about 200 units/day where the definition of a unit is an amount of PepF required to cleave one micromole of bradykinin at a pH of 8.0 and a temperature of 40° C.

In accordance with one or more further embodiments of the present invention, a medicament comprised of an effective dose or amount of subtilisin is administered to a patient to treat/cure Anorexia Nervosa. An effective dose or amount of subtilisin is a dose or amount of the protease that is effective in destroying or deactivating mimics, immunogens and/or antigens that cause or exacerbate Anorexia Nervosa. The effective amount of subtilisin administered will depend upon the severity of the disease process, but will range between about 2,000 fibrinolytic units/day and about 10,000 fibrinolytic units/day. For example, the subtilisin will be administered in one or more, preferably three, doses daily of subtilisin. It is believed that hydrolytic processes of the subtilisin will act in two areas: the gut and the blood. In the gut, the subtilisin will destroy or deactivate potential Anorexia Nervosa mimics before they can reach a sufficient level for immune activation, and, in the blood, the subtilisin will destroy any Anorexia Nervosa immunogens and antigens that could interact with the immune system and prolong a clinical exacerbation.

Subtilisin enzymes that are useful for treating Anorexia Nervosa are subtilisins from any of the subtilisin proteases included in the subtilase superfamily, family subtilisin, and subgroup true subtilisins. For example, one or more embodiments of the present invention comprise administering subtilisin proteases in the “true subtilisin” subgroup which includes subtilisin BPN′, subtilisin Carlsberg and subtilisin NAT.

In accordance with one or more further embodiments of the present invention, a medicament comprised of an effective amount of a non-pathogenic microorganism and/or its spores (that are capable of providing PepF) is administered to a patient to treat/cure Anorexia Nervosa. In accordance with one or more such embodiments, the microorganism and spores include, for example and without limitation, Lactobacillus jensenii, Lactobacillus crispatus, Lactobacillus johnsonii, Lactobacillus plantarum, Lactobacillus helveticus, Lactobacillus amylolyticus, Lactobacillus salivarius, Lactobacillus ultunensis, Lactobacillus rhamnosus, Lactobacillus acidophilus, Lactobacillus delbrueki bulgaricus, Lactobacillus gasseri, Lactobacillus casei, Lactobacillus coleohominis, Lactobacillus fermentum, Lactobacillus paracasei, Lactococcus lactis cremoris, Enterococcus faecalis, Bacillus cereus (spore-forming), Campylobacter subtilisin, and Oenococcus oeni and their various strains. In accordance with one or more such embodiments, an effective dose or amount of the non-pathenogenic microorganism and/or its spores (that are capable of providing PepF) is an amount that is effective to cause destruction or deactivation of mimics, immunogens and/or antigens that cause or exacerbate Anorexia Nervosa. The effective dose administered will depend upon the severity of the disease process (the medicament will be administered one or more, preferably three, doses daily), but the amount of the microorganism and spores in the dose will range from about 100 thousand CFU to about 600 billion CFU per dose, where the dose is administered about one or more times per week, or as often as about one to about three times daily.

In accordance with one or more further embodiments of the present invention, a medicament comprised of an effective amount of a non-pathogenic microorganism and/or its spores (that are capable of providing subtilisin) is administered to a patient to treat/cure Anorexia Nervosa. In accordance with one or more such embodiments, the microorganism and spores include, for example and without limitation, Bacillus subtilis, Bacillus licheniformis, and Bacillus lentus and their various strains. In accordance with one or more such embodiments, an effective dose or amount of the non-pathenogenic microorganism and/or its spores (that are capable of providing subtilisin) is an amount that is effective to cause destruction or deactivation of mimics, immunogens and/or antigens that cause or exacerbate Anorexia Nervosa. The effective dose administered will depend upon the severity of the disease process (the medicament will be administered one or more, preferably three, doses daily), but the amount of the microorganism and spores in the dose will range from about 100 thousand CFU to about 600 billion CFU per dose.

In accordance with one or more further embodiments of the present invention, a medicament comprised of an effective amount of parts of, or entire broken up, microorganisms and/or their spores (that are capable of providing PepF) is administered to a patient to treat/cure Anorexia Nervosa. Parts of the microorganisms and/or spores can be separated and selected, using any one of a number of methods that are well known to those of ordinary skill in the art, for their bioactive properties to help ensure and improve the rate of the destruction or deactivation of mimics, immunogens and/or antigens that cause or exacerbate Anorexia Nervosa and/or to improve the effectiveness of enzymes in the gastrointestinal or respiratory tract in destroying or deactivating such mimics, immunogens and/or antigens. In accordance with one or more such embodiments, the microorganism and spores include, for example and without limitation, Lactobacillus jensenii, Lactobacillus crispatus, Lactobacillus johnsonii, Lactobacillus plantarum, Lactobacillus helveticus, Lactobacillus amylolyticus, Lactobacillus salivarius, Lactobacillus ultunensis, Lactobacillus rhamnosus, Lactobacillus acidophilus, Lactobacillus delbrueki bulgaricus, Lactobacillus gasseri, Lactobacillus casei, Lactobacillus coleohominis, Lactobacillus fermentum, Lactobacillus paracasei, Lactococcus lactis cremoris, Enterococcus faecalis, Bacillus cereus (spore-forming), Campylobacter subtilisin, and Oenococcus oeni and their various strains. In accordance with one or more such embodiments, an effective dose or amount of parts of, or entire broken up, microorganisms and/or their spores (that are capable of providing PepF) is an amount that is effective to cause destruction or deactivation of mimics, immunogens and/or antigens that cause or exacerbate Anorexia Nervosa. The effective dose administered will depend upon the severity of the disease process (the medicament will be administered one or more, preferably three, doses daily), but the amount of parts of, or entire broken up, microorganisms and/or their spores will range from about 100 thousand CFU to about 600 billion CFU per dose. Methods for breaking up suitable microorganisms and/or spores are set forth above with respect to MS.

In accordance with one or more further embodiments of the present invention, a medicament comprised of an effective amount of parts of, or entire broken up, microorganisms and/or their spores (that are capable of providing subtilisin) is administered to a patient to treat/cure Anorexia Nervosa. Parts of the microorganisms and/or spores can be separated and selected, using any one of a number of methods that are well known to those of ordinary skill in the art, for their bioactive properties to help ensure and improve the rate of the destruction or deactivation of mimics, immunogens and/or antigens that cause or exacerbate Anorexia Nervosa and/or to improve the effectiveness of enzymes in the gastrointestinal or respiratory tract in destroying or deactivating such mimics, immunogens and/or antigens. In accordance with one or more such embodiments, the microorganism and spores include, for example and without limitation, Bacillus subtilis, Bacillus licheniformis, and Bacillus lentus and their various strains. In accordance with one or more such embodiments, an effective dose or amount of parts of, or entire broken up, microorganisms and/or their spores (that are capable of providing subtilisin) is an amount that is effective to cause destruction or deactivation of mimics, immunogens and/or antigens that cause or exacerbate Anorexia Nervosa. The effective dose administered will depend upon the severity of the disease process (the medicament will be administered one or more, preferably three, doses daily), but the amount of parts of, or entire broken up, microorganisms and/or their spores will range from about 100 thousand CFU to about 600 billion CFU per dose. Methods for breaking up suitable microorganisms and/or spores are set forth above with respect to MS.

In accordance with one or more embodiments, the above-described medicaments can be administered: orally (the methods relating to oral administration described above with respect to MS may also be applied with respect to Anorexia Nervosa); rectally (the methods relating to rectal administration and selection of microorganisms as sources of suitable enzymes described above with respect to MS may also be applied with respect to Anorexia Nervosa); transdermally (the methods relating to transdermal administration described above with respect to MS may also be applied with respect to Anorexia Nervosa); intravenously (the methods relating to intravenous administration described above with respect to MS may also be applied with respect to Anorexia Nervosa); and by inhalation (the methods relating to inhalation administration described above with respect to MS may also be applied with respect to Anorexia Nervosa).

In accordance with one or more embodiments, a medicament comprised of PepF can be combined with other enzymes or probiotics such as, for example and without limitation, subtilisin or Lactobacillus lactis L1A.

Rheumatoid Arthritis: In accordance with one or more further embodiments of the present invention, a medicament comprised of an effective dose or amount of subtilisin is administered to a patient to treat/cure Rheumatoid Arthritis. An effective dose or amount of subtilisin is a dose or amount of the protease that is effective in destroying or deactivating mimics, immunogens and/or antigens that cause or exacerbate Rheumatoid Arthritis. The effective amount of subtilisin administered will depend upon the severity of the disease process, but will range between about 2,000 fibrinolytic units/day and about 10,000 fibrinolytic units/day. For example, the subtilisin will be administered in one or more, preferably three, doses daily of subtilisin. It is believed that hydrolytic processes of the subtilisin will act in two areas: the gut and the blood. In the gut, the subtilisin will destroy or deactivate potential Rheumatoid Arthritis mimics before they can reach a sufficient level for immune activation, and, in the blood, the subtilisin will destroy or deactivate Rheumatoid Arthritis immunogens and antigens released from cartilage that could interact with the immune system and prolong a clinical exacerbation.

Subtilisin enzymes that are useful for treating Anorexia Nervosa are subtilisins from any of the subtilisin proteases included in the subtilase superfamily, family subtilisin, and subgroup true subtilisins. For example, one or more embodiments of the present invention comprise administering subtilisin proteases in the “true subtilisin” subgroup which includes subtilisin BPN', subtilisin Carlsberg and subtilisin NAT.

In accordance with one or more further embodiments, a medicament comprised of an effective dose of endonuclease O (PepO) from Bifidobacterium animalis subsp lactis is administered to a patient to treat/cure Rheumatoid Arthritis. While this is a PepO endonuclease, the protease from Bifidobacterium has an ability to hydrolyze pro-pro bonds that has not been reported with other endopeptidase O proteases.

In accordance with one or more such embodiments, an effective dose or amount of PepO is a dose or amount of the protease (for example, in sufficient concentration) that is effective in destroying or deactivating mimics, immunogens antigens that cause or exacerbate Rheumatoid Arthritis. The effective amount of endopeptidase O (PepO) administered will depend upon the severity of the disease process (the medicament will be administered one or more, preferably three, doses daily), but will range between about 20 units/day and about 200 units/day where the definition of a unit is the amount of PepO from Bifdobacterium animalis subsp. lactis required to cleave one micromole of bradykinin at a pH of 6.0 and a temperature of 25° C.

In accordance with one or more further embodiments of the present invention, a medicament comprised of an effective dose of endopeptidase O2 (PepO2) is administered to a patient to treat/cure Rheumatoid Arthritis. In accordance with one or more such embodiments, an effective dose or amount of PepO2 is a dose or amount of the PepO2 (for example, in sufficient concentration) that is effective in destroying or deactivating mimics, immunogens antigens that cause or exacerbate Rheumatoid Arthritis. The effective amount of endopeptidase O2 (PepO2) administered will depend upon the severity of the disease process (the medicament will be administered one or more, preferably three, doses daily), but will range between about 20 units/day and about 200 units/day where the definition of a unit is an amount of PepO2 required to cleave one micromole of BCN (f193-209) at a pH of 6.5 and a temperature of 25° C.

In accordance with one or more further embodiments of the present invention, a medicament comprised of an effective amount of a non-pathogenic microorganism and/or its spores (that are capable of providing subtilisin) is administered to a patient to treat/cure Rheumatoid Arthritis. In accordance with one or more such embodiments, the microorganism and spores include, for example and without limitation, Bacillus subtilis, Bacillus licheniformis, and Bacillus lentus and their various strains. In accordance with one or more such embodiments, an effective dose or amount of the non-pathenogenic microorganism and/or its spores (that are capable of providing subtilisin) is an amount that is effective to cause destruction or deactivation of mimics, immunogens and/or antigens that cause or exacerbate Rheumatoid Arthritis. The effective dose administered will depend upon the severity of the disease process (the medicament will be administered one or more, preferably three, doses daily), but the amount of the microorganism and spores in the dose will range from about 100 thousand CFU to about 600 billion CFU per dose.

In accordance with one or more further embodiments of the present invention, a medicament comprised of an effective amount of a non-pathogenic microorganism and/or its spores (that are capable of providing PepO) is administered to a patient to treat/cure Rheumatoid Arthritis. In accordance with one or more such embodiments, the microorganism and spores include, for example and without limitation, Bifidobacterium animalis subsp lactis and their various strains. In accordance with one or more such embodiments, an effective dose or amount of the non-pathenogenic microorganism and/or its spores (that are capable of providing PepO) is an amount that is effective to cause destruction or deactivation of mimics, immunogens and/or antigens that cause or exacerbate Rheumatoid Arthritis. The effective dose administered will depend upon the severity of the disease process (the medicament will be administered one or more, preferably three, doses daily), but the amount of the microorganism and spores in the dose will range from about 100 thousand CFU to about 600 billion CFU per dose, where the dose is administered about one or more times per week, or as often as about one to about three times daily.

In accordance with one or more further embodiments of the present invention, a medicament comprised of an effective amount of a non-pathogenic microorganism and/or its spores (that are capable of providing PepO2) is administered to a patient to treat/cure Rheumatoid Arthritis. In accordance with one or more such embodiments, the microorganism and spores include, for example and without limitation, Lactococcus lactis cremoris, Lactobacillus helveticus, and Lactobacillus johnsonii and their various strains. In accordance with one or more such embodiments, an effective dose or amount of the non-pathenogenic microorganism and/or its spores (that are capable of providing PepO2) is an amount that is effective to cause destruction or deactivation of mimics, immunogens and/or antigens that cause or exacerbate Rheumatoid Arthritis. The effective dose administered will depend upon the severity of the disease process (the medicament will be administered one or more, preferably three, doses daily), but the amount of the microorganism and spores in the dose will range from about 100 thousand CFU to about 600 billion CFU per dose, where the dose is administered about one or more times per week, or as often as about one to about three times daily.

In accordance with one or more further embodiments of the present invention, a medicament comprised of an effective amount of parts of, or entire broken up, microorganisms and/or their spores (that are capable of providing subtilisin) is administered to a patient to treat/cure Rheumatoid Arthritis. Parts of the microorganisms and/or spores can be separated and selected, using any one of a number of methods that are well known to those of ordinary skill in the art, for their bioactive properties to help ensure and improve the rate of the destruction or deactivation of mimics, immunogens and/or antigens that cause or exacerbate Rheumatoid Arthritis and/or to improve the effectiveness of enzymes in the gastrointestinal or respiratory tract in destroying or deactivating such mimics, immunogens and/or antigens. In accordance with one or more such embodiments, the microorganism and spores include, for example and without limitation, Bacillus subtilis, Bacillus licheniformis, and Bacillus lentus. In accordance with one or more such embodiments, an effective dose or amount of parts of, or entire broken up, microorganisms and/or their spores (that are capable of providing subtilisin) is an amount that is effective to cause destruction or deactivation of mimics, immunogens and/or antigens that cause or exacerbate Rheumatoid Arthritis. The effective dose administered will depend upon the severity of the disease process (the medicament will be administered one or more, preferably three, doses daily), but the amount of parts of, or entire broken up, microorganisms and/or their spores will range from about 100 thousand CFU to about 600 billion CFU per dose. Methods for breaking up suitable microorganisms and/or spores are set forth above with respect to MS.

In accordance with one or more further embodiments of the present invention, a medicament comprised of an effective amount of parts of, or entire broken up, microorganisms and/or their spores (that are capable of providing PepO) is administered to a patient to treat/cure Rheumatoid Arthritis. Parts of the microorganisms and/or spores can be separated and selected, using any one of a number of methods that are well known to those of ordinary skill in the art, for their bioactive properties to help ensure and improve the rate of the destruction or deactivation of mimics, immunogens and/or antigens that cause or exacerbate Rheumatoid Arthritis and/or to improve the effectiveness of enzymes in the gastrointestinal or respiratory tract in destroying or deactivating such mimics, immunogens and/or antigens. In accordance with one or more such embodiments, the microorganism and spores include, for example and without limitation, Bifidobacterium animalis subsp lactis and their various strains. In accordance with one or more such embodiments, an effective dose or amount of parts of, or entire broken up, microorganisms and/or their spores (that are capable of providing PepO) is an amount that is effective to cause destruction or deactivation of mimics, immunogens and/or antigens that cause or exacerbate Rheumatoid Arthritis. The effective dose administered will depend upon the severity of the disease process (the medicament will be administered one or more, preferably three, doses daily), but the amount of parts of, or entire broken up, microorganisms and/or their spores will range from about 100 thousand CFU to about 600 billion CFU per dose. Methods for breaking up suitable microorganisms and/or spores are set forth above with respect to MS.

In accordance with one or more further embodiments of the present invention, a medicament comprised of an effective amount of parts of, or entire broken up, microorganisms and/or their spores (that are capable of providing PepO2) is administered to a patient to treat/cure Rheumatoid Arthritis. Parts of the microorganisms and/or spores can be separated and selected, using any one of a number of methods that are well known to those of ordinary skill in the art, for their bioactive properties to help ensure and improve the rate of the destruction or deactivation of mimics, immunogens and/or antigens that cause or exacerbate Rheumatoid Arthritis and/or to improve the effectiveness of enzymes in the gastrointestinal or respiratory tract in destroying or deactivating such mimics, immunogens and/or antigens. In accordance with one or more such embodiments, the microorganism and spores include, for example and without limitation, Lactococcus lactis cremoris, Lactobacillus helveticus, and Lactobacillus johnsonii and their various strains. In accordance with one or more such embodiments, an effective dose or amount of parts of, or entire broken up, microorganisms and/or their spores (that are capable of providing PepO) is an amount that is effective to cause destruction or deactivation of mimics, immunogens and/or antigens that cause or exacerbate Rheumatoid Arthritis. The effective dose administered will depend upon the severity of the disease process (the medicament will be administered one or more, preferably three, doses daily), but the amount of parts of, or entire broken up, microorganisms and/or their spores will range from about 100 thousand CFU to about 600 billion CFU per dose. Methods for breaking up suitable microorganisms and/or spores are set forth above with respect to MS.

In accordance with one or more embodiments, the above-described medicaments can be administered: orally (the methods relating to oral administration described above with respect to MS may also be applied with respect to Rheumatoid Arthritis); rectally (the methods relating to rectal administration and selection of microorganisms as sources of suitable enzymes described above with respect to MS may also be applied with respect to Rheumatoid Arthritis); transdermally (the methods relating to transdermal administration described above with respect to MS may also be applied with respect to Rheumatoid Arthritis); intravenously (the methods relating to intravenous administration described above with respect to MS may also be applied with respect to Rheumatoid Arthritis); and by inhalation (the methods relating to inhalation administration described above with respect to MS may also be applied with respect to Rheumatoid Arthritis).

In accordance with one or more embodiments, a medicament comprised of PepF can be combined with other enzymes or probiotics, and examples of other enzymes or probiotics such as, for example and without limitation, subtilisin or Lactobacillus lactis L1A.

Autistic Spectrum Disorder: Autistic Spectrum Disorder patients have gluten and casein sensitivities, molecules that are high in concentration of proline, and some autistic children have been found with antibodies to myelin basic protein. Thus, in accordance with one or more embodiments of the present invention, a medicament comprised of an effective dose or amount of Oenococcus oeni is administered to a patient to treat/cure Autistic Spectrum Disorder (referred to herein as Autism). It is believed that this treatment will help reduce the concentration of small hormonally active proteins produced from gluten digestion from contributing negatively biochemically, and will address a problem of clinical signs persisting and exacerbating after gluten immunogens have been destroyed. An effective dose or amount of Oenococcus oeni is a dose or amount of Oenococcus oeni that is effective in destroying or deactivating mimics, immunogens and/or antigens that cause or exacerbate Autistic Spectrum Disorder. The effective amount of Oenococcus oeni administered will depend upon the severity of the disease process, but each dose should contain from about 1×10⁵ to about 600×10⁹ CFU per dose of each strain, and preferably >1×10⁷ CFU per dose of each single strain.

In accordance with one or more further embodiments, a medicament comprised of an effective dose of endopeptidase O (PepO) or a combination of PepO and Oenococcus oeni will be administered to a patient to treat/cure Autistic Spectrum Disorder. PepO specifically hydrolyzes 20-5 amino acids peptides on the N-terminal side of hydrophobic amino acids, and known immunogens fall in this range. Hence, the PepO will destroy or help destroy mimics, immunogens and/or antigens for Autistic Spectrum Disorder, and it will also destroy or help destroy small biologically active proline peptides which adversely affect Autism patients. Thus, once digested, smaller peptides can be quickly absorbed and transported into bacterial cells for further destruction to individual amino acids. As a result, the concentration of small proline containing peptides, especially C-terminal proline peptides that can contribute to hormonal adversities, are reduced.

In accordance with one or more such embodiments, an effective dose or amount of PepO or a combination of PepO and Oenococcus oeni is a dose or amount (for example, in sufficient concentration) that is effective in destroying or deactivating mimics, immunogens antigens that cause or exacerbate Autistic Spectrum Disorder. The effective amount of PepO or a combination of PepO and Oenococcus oeni administered will depend upon the severity of the disease process (the medicament will be administered one or more, preferably three, doses daily), but the amount of the PepO in the dose will range between about 20 units/day and about 200 units/day where the definition of a unit is the amount of PepO required to cleave one micromole of bradykinin at a pH of 6.0 and a temperature of 25° C. and the amount of the Oenococcus oeni in a dose will range from about 1×10⁵ to about 600×10⁹ CFU per dose of each strain, and preferably >1×10⁷ CFU per dose of each single strain.

In accordance with one or more further embodiments, a medicament comprised of an effective dose of endopeptidase O2 (PepO2) or a combination of PepO2 and Oenococcus oeni is administered to a patient to treat/cure Autistic Spectrum Disorder. In accordance with one or more such embodiments, an effective dose or amount of endopeptidase O2 (PepO2) or a combination of PepO2 and Oenococcus oeni is a dose or amount (for example, in sufficient concentration) that is effective in destroying or deactivating mimics, immunogens antigens that cause or exacerbate Autistic Spectrum Disorder. The effective amount of endopeptidase O2 (PepO2) or a combination of PepO2 and Oenococcus oeni administered will depend upon the severity of the disease process (the medicament will be administered one or more, preferably three, doses daily), and the amount of the PepO2 in the dose will range between about 20 units/day and about 200 units/day where the definition of a unit is the amount of PepO2 required to cleave one micromole of BCN (f193-209) at a pH of 6.5 and a temperature of 25° C. and the amount of the Oenococcus oeni in the dose will range from about 1×10⁵ to about 600×10⁹ CFU per dose of each strain, and preferably >1×10⁷ CFU per dose of each single strain.

In accordance with one or more further embodiments, a medicament comprised of an effective dose of endopeptidase F (PepF) or a combination of PepF and Oenococcus oeni is administered to a patient to treat/cure Autistic Spectrum Disorder. This enzyme, also a metallopeptidase, hydrolyzes 21-5 (for Bacillus amyloliquefaciens PepF; see, for example, an article entitled “Characterization of a novel Pep-F-like oligopeptidase secreted by Bacillus amyloliquefaciens” by S.-H Chao, T.-H. Cheng, C.-Y. Shaw, M. H. Lee, Y.-H. Hsu and Y.-C. Tsai in 23-7A. App. Environ. Microbiol., 72 pp. 968-971 (2006)) and 17-7 (for Lactococcus lactis; see, for example, an article entitled “Biochemical and Genetic Characterization of PepF, an Oligopeptidase from Lactococcus Lactis” by V. Monnet, M. Nardi, A. Chopin, M.-C. Chopin and J.-C. Gripon in J. Biol. Chem., 269 pp. 32070-32076 (1994)) amino acid peptides on the N-terminal side of hydrophobic amino acids. Its activity is similar to PepO, but it is less active toward proline bonds. The enzyme has also been reported in Bacillus subtilis.

In accordance with one or more such embodiments, an effective dose or amount of endopeptidase F (PepF) or a combination of PepF and Oenococcus oeni is a dose or amount (for example, in sufficient concentration) that is effective in destroying or deactivating mimics, immunogens antigens that cause or exacerbate Autistic Spectrum Disorder. The effective amount of an effective dose or amount of PepF or a combination of PepF and Oenococcus oeni administered will depend upon the severity of the disease process (the medicament will be administered one or more, preferably three, doses daily), and the amount of the PepF in the dose will range between about 20 units/day and about 200 units/day where the definition of a unit is the amount of PepF required to cleave one micromole of bradykinin at a pH of 8.0 and a temperature of 40° C. and the amount of the Oenococcus oeni in the dose will range from about 1×10⁵ to about 600×10⁹ CFU per dose of each strain, and preferably >1×10⁷ CFU per dose of each single strain.

In accordance with one or more further embodiments of the present invention, a medicament comprised of an effective amount of a non-pathenogenic microorganism and/or its spores (that are capable of providing PepO) or a combination of Oenococcus oeni and a non-pathenogenic microorganism and/or its spores (that are capable of providing PepO) is administered to a patient to treat/cure Autistic Spectrum Disorder. In accordance with one or more such embodiments, the microorganism and spores include, for example and without limitation, Lactobacillus sanfrancisens, Lactobacillus plantarum, Lactobacillus acidophilus, Lactobacillus helveticus, Lactobacillus casei, Lactobacillus reuteri, Lactobacillus salivarius, Lactobacillus gasseri, Lactobacillus rhamnosus, Lactobacillus johnsonii, Lactobacillus jensenii, Lactobacillus amylolyticus, Lactobacillus sakei, Lactobacillus antri, Lactobacillus paracasei, Lactobacillus ruminis, Lactococcus lactis, Bifidobacterium dentium, Bifidobacterium longum, Bifidobacterium adolescentis, Bifidobacterium animalis, and Oenicoccus oeni and their various strains. In accordance with one or more such embodiments, an effective dose or amount of the non-pathenogenic microorganism and/or its spores (that are capable of providing PepO) or a combination of Oenococcus oeni and a non-pathenogenic microorganism and/or its spores (that are capable of providing PepO) is an amount that is effective to cause destruction or deactivation of mimics, immunogens and/or antigens that cause or exacerbate Autistic Spectrum Disorder. The effective dose administered will depend upon the severity of the disease process (the medicament will be administered one or more, preferably three, doses daily), but the amount of the microorganism and spores in the dose will range from about 100 thousand CFU to about 600 billion CFU per dose, and the amount of the Oenococcus oeni in a dose will range from about 1×10⁵ to about 600×10⁹ CFU per dose of each strain, and preferably >1×10⁷ CFU per dose of each single strain.

In accordance with one or more further embodiments of the present invention, a medicament comprised of an effective amount of a non-pathenogenic microorganism and/or its spores (that are capable of providing PepO2) or a combination of Oenococcus oeni and a non-pathenogenic microorganism and/or its spores (that are capable of providing PepO2) is administered to a patient to treat/cure Autistic Spectrum Disorder. In accordance with one or more such embodiments, the microorganism and spores include, for example and without limitation, Lactococcus lactis cremoris, Lactobacillus helveticus, and Lactobacillus johnsonii and their various strains. In accordance with one or more such embodiments, an effective dose or amount of the non-pathenogenic microorganism and/or its spores (that are capable of providing PepO2) or a combination of Oenococcus oeni and a non-pathenogenic microorganism and/or its spores (that are capable of providing PepO2) is an amount that is effective to cause destruction or deactivation of mimics, immunogens and/or antigens that cause or exacerbate Autistic Spectrum Disorder. The effective dose administered will depend upon the severity of the disease process (the medicament will be administered one or more, preferably three, doses daily), but the amount of the microorganism and spores in the dose will range from about 100 thousand CFU to about 600 billion CFU per dose, and the amount of the Oenococcus oeni in a dose will range from about 1×10⁵ to about 600×10⁹ CFU per dose of each strain, and preferably >1×10⁷ CFU per dose of each single strain.

In accordance with one or more further embodiments of the present invention, a medicament comprised of an effective amount of a non-pathenogenic microorganism and/or its spores (that are capable of providing PepF) or a combination of Oenococcus oeni and a non-pathenogenic microorganism and/or its spores (that are capable of providing PepF) is administered to a patient to treat/cure Autistic Spectrum Disorder. In accordance with one or more such embodiments, the microorganism and spores include, for example and without limitation, but not limited to, Lactobacillus jensenii, Lactobacillus crispatus, Lactobacillus johnsonii, Lactobacillus plantarum, Lactobacillus helveticus, Lactobacillus amylolyticus, Lactobacillus salivarius, Lactobacillus ultunensis, Lactobacillus rhamnosus, Lactobacillus acidophilus, Lactobacillus delbrueki bulgaricus, Lactobacillus gasseri, Lactobacillus casei, Lactobacillus coleohominis, Lactobacillus fermentum, Lactobacillus paracasei, Lactococcus lactis cremoris, Enterococcus faecalis, Bacillus cereus (spore-forming), Campylobacter subtilisin, and Oenococcus oeni and their various strains. In accordance with one or more such embodiments, an effective dose or amount of the non-pathenogenic microorganism and/or its spores (that are capable of providing PepF) or a combination of Oenococcus oeni and a non-pathenogenic microorganism and/or its spores (that are capable of providing PepF) is an amount that is effective to cause destruction or deactivation of mimics, immunogens and/or antigens that cause or exacerbate Autistic Spectrum Disorder. The effective dose administered will depend upon the severity of the disease process (the medicament will be administered one or more, preferably three, doses daily), but the amount of the microorganism and spores in the dose will range from about 100 thousand CFU to about 600 billion CFU per dose, and the amount of the Oenococcus oeni in a dose will range from about 1×10⁵ to about 600×10⁹ CFU per dose of each strain, and preferably >1×10⁷ CFU per dose of each single strain.

In accordance with one or more further embodiments of the present invention, a medicament comprised of an effective amount of parts of, or entire broken up, microorganisms and/or their spores (that are capable of providing PepO) or a combination of Oenococcus oeni parts of, or entire broken up, microorganisms and/or their spores (that are capable of providing PepO) is administered to a patient to treat/cure Autistic Spectrum Disorder. Parts of the microorganisms and/or spores can be separated and selected, using any one of a number of methods that are well known to those of ordinary skill in the art, for their bioactive properties to help ensure and improve the rate of the destruction or deactivation of mimics, immunogens and/or antigens that cause or exacerbate Autistic Spectrum Disorder and/or to improve the effectiveness of enzymes in the gastrointestinal or respiratory tract in destroying or deactivating such mimics, immunogens and/or antigens. In accordance with one or more embodiments of this invention, the parts of the microorganisms and/or spores chosen can be, but are not limited to, adjuvants such as muramyl dipeptide. The adjuvant's content can be increased or decreased to saturate or starve the gastrointestinal tract or respiratory tract of the adjuvant to prevent the optimum ratio of adjuvant to mimic from being close enough to one to initiate and/or maintain the autoimmune reaction. In accordance with one or more such embodiments, the microorganism and spores include, for example and without limitation, Lactobacillus sanfrancisens, Lactobacillus plantarum, Lactobacillus acidophilus, Lactobacillus helveticus, Lactobacillus casei, Lactobacillus reuteri, Lactobacillus salivarius, Lactobacillus gasseri, Lactobacillus rhamnosus, Lactobacillus johnsonii, Lactobacillus jensenii, Lactobacillus amylolyticus, Lactobacillus sakei, Lactobacillus antri, Lactobacillus paracasei, Lactobacillus ruminis, Lactococcus lactis, Bifidobacterium dentium, Bifidobacterium longum, Bifidobacterium adolescentis, Bifidobacterium animalis, and Oenicoccus oeni and their various strains. In accordance with one or more such embodiments, an effective dose or amount of parts of, or entire broken up, microorganisms and/or their spores (that are capable of providing PepO) or a combination of Oenococcus oeni and parts of, or entire broken up, microorganisms and/or their spores (that are capable of providing PepO) is an amount that is effective to cause destruction or deactivation of mimics, immunogens and/or antigens that cause or exacerbate Autistic Spectrum Disorder. The effective dose administered will depend upon the severity of the disease process (the medicament will be administered one or more, preferably three, doses daily), but the amount of parts of, or entire broken up, microorganisms and/or their spores will range from about 100 thousand CFU to about 600 billion CFU per dose, and the amount of the Oenococcus oeni in a dose will range from about 1×10⁵ to about 600×10⁹ CFU per dose of each strain, and preferably >1×10⁷ CFU per dose of each single strain. Methods for breaking up suitable microorganisms and/or spores are set forth above with respect to MS.

In accordance with one or more further embodiments of the present invention, a medicament comprised of an effective amount of parts of, or entire broken up, microorganisms and/or their spores (that are capable of providing PepO2) or a combination of Oenococcus oeni and parts of, or entire broken up, microorganisms and/or their spores (that are capable of providing PepO2) is administered to a patient to treat/cure Autistic Spectrum Disorder. Parts of the microorganisms and/or spores can be separated and selected, using any one of a number of methods that are well known to those of ordinary skill in the art, for their bioactive properties to help ensure and improve the rate of the destruction or deactivation of mimics, immunogens and/or antigens that cause or exacerbate Autistic Spectrum Disorder and/or to improve the effectiveness of enzymes in the gastrointestinal or respiratory tract in destroying or deactivating such mimics, immunogens and/or antigens. In accordance with one or more such embodiments, the microorganism and spores include, for example and without limitation, Lactococcus lactis cremoris, Lactobacillus helveticus, and Lactobacillus johnsonii and their various strains. In accordance with one or more such embodiments, an effective dose or amount of parts of, or entire broken up, microorganisms and/or their spores (that are capable of providing PepO2) or a combination of Oenocooccus oeni and parts of, or entire broken up, microorganisms and/or their spores (that are capable of providing PepO2) is an amount that is effective to cause destruction or deactivation of mimics, immunogens and/or antigens that cause or exacerbate Autistic Spectrum Disorder. The effective dose administered will depend upon the severity of the disease process (the medicament will be administered one or more, preferably three, doses daily), but the amount of parts of, or entire broken up, microorganisms and/or their spores will range from about 100 thousand CFU to about 600 billion CFU per dose, and the amount of the Oenococcus oeni in a dose will range from about 1×10⁵ to about 600×10⁹ CFU per dose of each strain, and preferably >1×10⁷ CFU per dose of each single strain. Methods for breaking up suitable microorganisms and/or spores are set forth above with respect to MS.

In accordance with one or more further embodiments of the present invention, a medicament comprised of an effective amount of parts of, or entire broken up, microorganisms and/or their spores (that are capable of providing PepF) or a combination of Oenococcus oeni and parts of, or entire broken up, microorganisms and/or their spores (that are capable of providing PepF) is administered to a patient to treat/cure Autistic Spectrum Disorder. Parts of the microorganisms and/or spores can be separated and selected, using any one of a number of methods that are well known to those of ordinary skill in the art, for their bioactive properties to help ensure and improve the rate of the destruction or deactivation of mimics, immunogens and/or antigens that cause or exacerbate Autistic Spectrum Disorder and/or to improve the effectiveness of enzymes in the gastrointestinal or respiratory tract in destroying or deactivating such mimics, immunogens and/or antigens. In accordance with one or more such embodiments, the microorganism and spores include, for example and without limitation, Lactobacillus jensenii, Lactobacillus crispatus, Lactobacillus johnsonii, Lactobacillus plantarum, Lactobacillus helveticus, Lactobacillus amylolyticus, Lactobacillus salivarius, Lactobacillus ultunensis, Lactobacillus rhamnosus, Lactobacillus acidophilus, Lactobacillus delbrueki bulgaricus, Lactobacillus gasseri, Lactobacillus casei, Lactobacillus coleohominis, Lactobacillus fermentum, Lactobacillus paracasei, Lactococcus lactis cremoris, Enterococcus faecalis, Bacillus cereus (spore-forming), and Oenococcus oeni and their various strains. In accordance with one or more such embodiments, an effective dose or amount of parts of, or entire broken up, microorganisms and/or their spores (that are capable of providing PepF) or a combination of Oenococcus oeni and parts of, or entire broken up, microorganisms and/or their spores (that are capable of providing PepF) is an amount that is effective to cause destruction or deactivation of mimics, immunogens and/or antigens that cause or exacerbate Autistic Spectrum Disorder. The effective dose administered will depend upon the severity of the disease process (the medicament will be administered one or more, preferably three, doses daily), but the amount of parts of, or entire broken up, microorganisms and/or their spores will range from about 100 thousand CFU to about 600 billion CFU per dose, and the amount of the Oenococcus oeni in a dose will range from about 1×10⁵ to about 600×10⁹ CFU per dose of each strain, and preferably >1×10⁷ CFU per dose of each single strain. Methods for breaking up suitable microorganisms and/or spores are set forth above with respect to MS.

In accordance with one or more further embodiments of the present invention, a medicament comprised of an effective dose or amount of subtilisin is administered to a patient to treat/cure Autistic Spectrum Disorder. An effective dose or amount of subtilisin is a dose or amount of the protease that is effective in destroying or deactivating mimics, immunogens and/or antigens that cause or exacerbate Autistic Spectrum Disorder. The effective amount of subtilisin administered will depend upon the severity of the disease process, but will range between about 2,000 fibrinolytic units/day and about 10,000 fibrinolytic units/day. For example, the subtilisin will be administered in one or more, preferably three, doses daily of subtilisin. It is believed that hydrolytic processes of the subtilisin will act in two areas: the gut and the blood. In the gut, the subtilisin will destroy or deactivate potential Autistic Spectrum Disorder mimics before they can reach a sufficient level for immune activation, and, in the blood, the subtilisin will destroy any Autistic Spectrum Disorder immunogens and antigens that could interact with the immune system and prolong a clinical exacerbation.

Subtilisin enzymes that are useful for treating Autistic Spectrum Disorder are subtilisins from any of the subtilisin proteases included in the subtilase superfamily, family subtilisin, and subgroup true subtilisins. For example, one or more embodiments of the present invention comprise administering subtilisin proteases in the “true subtilisin” subgroup which includes subtilisin BPN', subtilisin Carlsberg and subtilisin NAT.

In accordance with one or more further embodiments of the present invention, a medicament comprised of an effective amount of a non-pathogenic microorganism and/or its spores (that are capable of providing subtilisin) is administered to a patient to treat/cure Autistic Spectrum Disorder. In accordance with one or more such embodiments, the microorganism and spores include, for example and without limitation, Bacillus subtilis, Bacillus licheniformis, and Bacillus lentus and their various strains. In accordance with one or more such embodiments, an effective dose or amount of the non-pathenogenic microorganism and/or its spores (that are capable of providing subtilisin) is an amount that is effective to cause destruction or deactivation of mimics, immunogens and/or antigens that cause or exacerbate Autistic Spectrum Disorder. The effective dose administered will depend upon the severity of the disease process (the medicament will be administered one or more, preferably three, doses daily), but the amount of the microorganism and spores in the dose will range from about 100 thousand CFU to about 600 billion CFU per dose.

In accordance with one or more further embodiments of the present invention, a medicament comprised of an effective amount of parts of, or entire broken up, microorganisms and/or their spores (that are capable of providing subtilisin) is administered to a patient to treat/cure Autistic Spectrum Disorder. Parts of the microorganisms and/or spores can be separated and selected, using any one of a number of methods that are well known to those of ordinary skill in the art, for their bioactive properties to help ensure and improve the rate of the destruction or deactivation of mimics, immunogens and/or antigens that cause or exacerbate Autistic Spectrum Disorder and/or to improve the effectiveness of enzymes in the gastrointestinal or respiratory tract in destroying or deactivating such mimics, immunogens and/or antigens. In accordance with one or more such embodiments, the microorganism and spores include, for example and without limitation, Bacillus subtilis, Bacillus licheniformis, and Bacillus lentus and their various strains. In accordance with one or more such embodiments, an effective dose or amount of parts of, or entire broken up, microorganisms and/or their spores (that are capable of providing subtilisin) is an amount that is effective to cause destruction or deactivation of mimics, immunogens and/or antigens that cause or exacerbate Autistic Spectrum Disorder. The effective dose administered will depend upon the severity of the disease process (the medicament will be administered one or more, preferably three, doses daily), but the amount of parts of, or entire broken up, microorganisms and/or their spores will range from about 100 thousand CFU to about 600 billion CFU per dose. Methods for breaking up suitable microorganisms and/or spores are set forth above with respect to MS.

In accordance with one or more embodiments, the above-described medicaments can be administered: orally (the methods relating to oral administration described above with respect to MS may also be applied with respect to Autistic Spectrum Disorder); rectally (the methods relating to rectal administration and selection of microorganisms as sources of suitable enzymes described above with respect to MS may also be applied with respect to Autistic Spectrum Disorder); transdermally (the methods relating to transdermal administration described above with respect to MS may also be applied with respect to Autistic Spectrum Disorder); intravenously (the methods relating to intravenous administration described above with respect to MS may also be applied with respect to Autistic Spectrum Disorder); and by inhalation (the methods relating to inhalation administration described above with respect to MS may also be applied with respect to Autistic Spectrum Disorder).

The following sets forth, for example and without limitation, methods for preparing useful probiotic microorganisms. Fermentation: As an example, microorganism Bacillus subtilis Natto produces the endoprotease subtilisin. Fermentation additives may be added to a culture of the microorganisms to enhance: production of microorganisms, ability of the microorganisms to survive in the gastrointestinal tract, ability of the microorganisms to adhere to the gastrointestinal tract, ability of the microorganisms to secrete desired proteases, ability of the microorganisms to secrete chemicals to enhance survival of proteases, ability of the microorganisms to secrete chemicals to enhance effectiveness of desired proteases, and ability of the microorganisms to secrete chemicals to interfere with undesired chemicals. Also, the amount and kinds of sugars, vitamins, amino acids, proteins and/or fats available to the microorganisms, prior to drying and forming a powder, affect their viability. Examples of useful sugars are, but are not limited to, sucrose, fructose, glucose, lactose, trehalose, raffinose, paliainose, lactulose, lactitol, xylitol, sorbitol, mannitol, malstose, dextrin and maltodextrin. Examples of useful anti-oxidants are, but are not limited to, ascorbic acid, glutathione and alpha-lipoic acid. Examples of useful amino acids or their salts are, but are not limited to, lysine, cysteine, glycine and glutamate. Examples of useful oils are, but are not limited to, butter, palm oil, nut oil, cocoa oil, rapeseed oil and soy bean oil. Examples of useful stabilizing ingredients are, but are not limited to, soybean oligosaccharides, frutooligosaccharides, galactooligosaccharides, galactosyl lactose, milk, milk powders, whey, whey protein concentrates, casein, casein hydrolysates, lactoferrin, lactoperoxidase, lactoglobulins, glymacropeptides, lacto-saccharides, glycomacropeptides, lacto-saccharides and lacto-lipids.

A chemical that inactivates an enzyme is, for example and without limitation, a serpin. Thus, it is desirable to inhibit serpins that inactivate proteases that destroy mimics, antigens, or immunogens that cause autoimmune disease. Also, protective agents such as, for example and without limitation, cryoprotectants or other chemicals such as gels, starches, polysaccharides, and/or sugars can be added to the culture to protect the microorganisms during the manufacturing processes.

Removing Liquid: When a powdered form of a probiotic is required, the microorganisms need to have liquid removed. One option, known to those of ordinary skill in the art, is to centrifuge the fermentation mixture to reduce the amount of liquid. Other options include, but are not limited to, settling and membrane filtration. This concentrates the microbes by separating them from their supernatant. As such, it allows for more microbes per kilogram of dried product to keep transportation and storage costs down and to deliver more microbes per capsule or pill. It also allows the microbes to be added as a concentrated powder to be mixed into a drink or sprinkled onto food. Centrifuging is beneficial when the supernatant does not contain substantial amounts of bioactive substances that the microbes cannot readily secrete or create in the gastrointestinal tract. However, if the supernatant has the bioactive substances for therapeutic effect, then centrifuging does not need to be performed if the microbes are still desired. In some cases, centrifuging or other separation processes, known to those of ordinary skill in the art, may be desired to obtain the bioactive substances, such as enzymes or bacteriocins, for delivery without the microbe. The following methods may also be used to separate bugs from supernatant: sedimentation, ultrafiltration and reverse osmosis.

Drying: This step dries the microorganisms as well as any available supernatant. The microorganisms can be dried with freeze-drying techniques, known to those of ordinary skill in the art, by placing the centrifuged microbes and residual supernatant, which form a slurry, onto trays and freezing them in a vacuum environment. After the slurry dries, it resembles a cake. The dried cake is then crushed, and the crushed powders are sieved to obtain the desired particle size distribution. This process of drying in bulk followed by crushing often kills many bacteria due to the thermal and mechanical stresses applied to the microbes. Another method of making a powder, known by those of ordinary skill in the art, is to spray dry the microorganisms. For this process the slurry is sprayed through a nozzle into a heated air environment. The incoming slurry can be heated or unheated. If the shear forces and temperatures that the microorganisms and/or enzymes experience during the heated spray drying process are too great, microorganisms will die or be damaged enough that the intended therapeutic effectiveness of the microorganism and/or enzyme will be diminished. To improve the yield and prevent damage to surviving microorganisms, an electrospray drying process can be used. Examples of manufacturers who make suitable electrospray drying equipment are Charge Injection Technology and Zoom Essence.

Blending: Other ingredients, such as but not limited to, dried proteases, microorganisms, protective sugars, polysaccharides, gums, oils, desiccants, anti-oxidants, and bacteriocins, can be added prior to or after the drying process. These ingredients will assist the microbes in surviving during storage as well as in passing to the target areas of the gastrointestinal tract. Also adding other ingredients is needed to reduce the dosage of concentrated microbial powders to the dosages required for delivery to the person.

Delivery: A powder can also be an acceptable delivery system especially when microbes do not need to be alive and where either their microbial parts or their secreted bioactive substances are effective against pathogens or for normalizing the protease ratios. The powder can be consumed by adding the powder to a food or drink product. The powder containing the microorganisms and/or enzymes can be formulated by those of ordinary skill in the art into a drink that may contain for example, but not limited to, water, sweeteners, flavorings, colorants, anti-oxidants, vitamins, minerals, short-chain fatty acids, stimulants, mood-enhancers, teas, anti-inflammatories, and other bioactive ingredients. The powder can be consumed after sprinkling or pouring it over solid food or mixing into a liquid. The powder can be packaged into bulk containers such as a bag or can or into individual sachets for easy of carrying and single use dosing. To form a tablet, a powder containing the microbes and/or enzyme(s), excipients, and/or other bioactive substances are compressed into a mold in a tableting machine. The tablet can be coated with methods and processes known to those of ordinary skill in the art to prevent the premature dissolution of the product in the stomach to keep the microbes alive for delivery further down the gastrointestinal (GI) tract. Such coatings are designed by those of ordinary skill in the art to dissolve by time in the GI tract or more preferably by pH exposure as the pH along the GI tract is acidic in the stomach and the pH increases by the time the digested contents reach the large intestine. At the large intestine, the pH is approximately 7. Some examples known to those of ordinary skill in the art of a pH triggered coating are, but not limited to, Eudragit and shellac. The tablet can be designed by those of ordinary skill in the art to be consumed orally or inserted into the rectum or vagina as a suppository. To form a capsule, a powder containing the microorganisms, enzymes, excipients and/or other bioactive substances are directed into a capsule that can be made of materials known to those of ordinary skill in the art, but are not limited to, hardened gelatin or other polymer. The capsule can be coated by processes known to those of ordinary skill in the art to prevent the premature dissolution of the product in the stomach to keep the microorganisms alive and enzymes effective for delivery further down the GI tract. Such coatings known to those who are of ordinary skill in the art are designed to dissolve by time in the GI tract or more preferably by pH exposure. Some examples known to those of ordinary skill in the art of a pH triggered coating are, but not limited to, Eudragit and shellac. The capsule can be designed by those of ordinary skill in the art to be consumed orally or inserted into the rectum or vagina as a suppository. An alternate form of a capsule to contain the microorganisms, enzymes, excipients, and/or other bioactive substances is a gel capsule that can be made of materials and processes known to those of ordinary skill in the art.

For a liquid delivery system, the microorganisms and/or enzymes and bioactive substances can be introduced simply in a fermented liquid. That liquid can be in the form of cultured or non-cultured animal-based and/or plant-based milk such as, but not limited to, cow's, goat's, rice, almond, and/or soy milk. Alternatively, microorganisms and/or enzymes can added to a drink such, as but not limited to, a juice or formulated into a drink that may contain for example but not limited to water, sweeteners, flavorings, colorants, anti-oxidants, vitamins, minerals, short-chain fatty acids, stimulants, mood-enhancers, teas, anti-inflammatories and other bioactive ingredients. For a solid delivery system the microorganisms and/or enzymes and bioactive substances can be added to solid food in accordance with a number of methods that are well known to those of ordinary skill in the art. Examples of such food are, but are not limited to, candy, confectionary, chewing gum, energy bars, fermented/dried vegetables, fermented/dried meat, fermented/dried seafood, fermented/dried fruit, fermented/dried beans and frozen desserts. For a slurry delivery system the microorganisms and/or enzymes and bioactive substances can be added to slurry foods in accordance with a number of methods that are well known to those of ordinary skill in the art. Examples of such food are, but not limited to, yogurt, jams, jellies, gravies, gel shots, puddings, frozen desserts, salad dressings, syrups and spreads.

Embodiments of the present invention described above are exemplary, and many changes and modifications may be made to the description set forth above by those of ordinary skill in the art while remaining within the scope of the invention. For example, the amino acid sequence of each peptidase discussed above will vary with each strain of the bacterial types discussed above. However, the mere changing of the amino acid sequence does not necessarily alter its biological properties and eliminate it from a particular proteolytic class. As such, the scope of the invention should be determined with reference to the appended claims along with their full scope of equivalents.

APPENDIX I
Table 1.
Chemically Defined Autoimmune Disease-Producing Immunogens

| | Description | Sequence |
|---|---|---|
| a. | Tryptophan peptide from myelin basic | PHE SER TRP GLY |
| | protein (EAE) (see an article entitled | ALA GLU GLY GLN |
| | “Essential Chemical Requirements | ARG |
| | for Induction of Allergic | |
| | Encephalomyelitis” by F. C. Westall, | |
| | A. B. Robinson, J. Caccam, | |
| | J. J. Jackson and E. H. Eylar | |
| | in Nature, 229 pp. 22-24 (1971)) | |
| b. | Mid region from myelin basic | THR THR HIS TYR |
| | protein (EAE) (see an article | GLY SER LEU PRO |
| | entitled “Biological Activity and | GLN LYS |
| | Synthesis of an Encephalitogenic | |
| | Determinant” by R. F. Shapira, | |
| | C. H. Chou, S. Mc Kneally, E. | |
| | Urban and R. F. Kibler in Science, | |
| | 172 pp. 736-738 (1971)) | |
| c. | Hyperacute site from myelin basic | PRO GLN LYS SER |
| | protein (EAE) (see an article | GLN ARG THR GLN |
| | entitled “Hyperacute Autoimmune | ASP GLU ASN PRO |
| | Encephalomyelitis-Unique Determinant | VAL |
| | Conferred by Serine in a Synthetic | |
| | Autoantigen” by F. C. Westall, | |
| | M. Thompson and V. A. Lennon | |
| | in Nature, 269 pp. 425-427 (1977)) | |
| d. | S-antigen 375-386 (see an article entitled | PHE VAL PHE GLU |
| | “Structure-function studies of s-antigen: | GLU PHE ALA ARG |
| | use of proteases to reveal a dominant | GLN ASN LEU LYS |
| | uveitogenic site” by H. S. Dua, | |
| | P. Hossain, P. A. J. Brown, A. McKinnon, | |
| | J. V. Forrester, S. Gregerson and | |
| | L. A. Donoso in Autoimmunity, | |
| | 10 pp. 153-163 (1991)) (EAU) | |
| e. | S-antigen 352-364 (EAU) | PRO PHE ARG LEU |
| | (see an article entitled “Identification | MET HIS PRO GLN |
| | of a Potent New Pathogenic Site in | PRO GLU ASP PRO |
| | Human Retinal S-Antigen which Induces | ASP |
| | Experimental Autoimmune Uveoretinitis | |
| | in LEW Rats” by D. S. Gregerson, | |
| | C. F. Merryman, W. F. Obritsch | |
| | and L. A. Donoso in Cell. Immunol., | |
| | 128 pp. 209-219 (1990)) | |
| f. | Interphotoreceptor retinoid | TYR ILE ILE SER |
| | binding protein (IRBP)165-177 (EAU) | TYR LEU HIS PRO |
| | (see an article entitled “Identification of a | GLY ASN THR ILE |
| | Major Pathogenic Epitope in the Human | LEU |
| | IRBP Molecule Recognized by | |
| | Mice of the H-2 Hapotype” by P. B. | |
| | Silver, L. V. Rizzo, C.-C. Chan, | |
| | L. A. Donoso, B. Wiggert and | |
| | R. R. Caspi in Invest. Ophthalmol. | |
| | Vis. Sci., 36 pp. 946-954 (1995)) | |
| g. | Interphotoreceptor retinoid binding | PHE GLN PRO SER |
| | protein 5-20 (see an article entitled | LEU VAL LEU ASP |
| | “Identification of a new epitope of | MET ALA LYS VAL |
| | human IRBP that induces | LEU LEU ASP |
| | autoimmune uveoretinitis in mice | |
| | of the H-2b haplotype” by D. Avichezer, | |
| | P. B. Silver, C. -C. Chan, B. Wiggert | |
| | and R. R. Caspi in Invest. Opthalmol. | |
| | Vis. Sci., 41 pp. 127-131 (2000)) (EAU) | |
| h. | Interphotoreceptor retinoid binding | ALA ASP LYS ASP |
| | protein 201-216 (EAU) (see an article | VAL VAL LEU THR |
| | entitled “Identification of a Peptide | SER SER ARG THR |
| | Inducing Experimental Autoimmune | GLY GLY VAL |
| | Uveoretinitis in H2Ak-Carrying Mice” | |
| | by K. Namba, K. Ogasawara, N. Kitaichi, | |
| | A. Matsuki et al. in Clin. Exp. Immunol., | |
| | 111 pp. 442-449 (1998). | |
| i. | Acetylcholine receptor 129-145 (EMG) | GLU ILE ILE VAL |
| | (see an article entitled “A 17-Mer Self- | THR HIS PHE PRO |
| | Peptide of Acetylcholine Receptor | PHE ASP GLU GLN |
| | Binds to B Cell MHC Class II. | ASN CYS SER MET |
| | Activates Helper T Cells, Stimulates | LYS |
| | Autoantibody Production and | |
| | electrophysiologic signs of myasthenia | |
| | gravis” by H. Yoshikawa, E. H. Lambert, | |
| | D. R. Walser-Kuntz, Y. Yasukawa, D. J. | |
| | Mc Cormick and V. A. Lennon in | |
| | J. Immunol., 159 pp. 1570-1577 (1997)) | |
| j. | Acetylcholine receptor 67-75 (EMG) | TRP ASN PRO ASP |
| | (see an article entitled “The Main Region | ASP TYR GLY GLY |
| | of the Nicotinic Acetylcholine Receptor” | VAL LYS |
| | by M. Bellone, F. Tang, R. Milius and | |
| | B. M. Conti-Troncoi in J. Immunol., | |
| | 143 pp. 3568-3579 (1989)) | |
| k. | Acetylcholine receptor 97-116 (EMG) | ASP GLY ASP PHE |
| | (see an article entitled “The Main Region | ALA ILE VAL LYS |
| | of the Nicotinic Acetylcholine Receptor” | PHE THR LYS VAL |
| | by M. Bellone, F. Tang, R. Milius and | LEU LEU ASP TYR |
| | B. M. Conti-Troncoi in J. Immunol., | THR GLY HIS ILE |
| | 143 pp. 3568-3579 (1989)) | |
| 1. | Acetylcholine receptor 195-212 (EMG) | ASP THR PRO TYR |
| | (see an article entitled “Effect of T cell | LEU ASP ILE THR |
| | recognition and immunogenicity of | TYR HIS PHE VAL |
| | alanine-substituted peptides | MET GLN ARG LEU |
| | corresponding to 97-116 sequence of the | PRO LEU |
| | rat AChR alpha-subunit” by F. Baggi, | |
| | A. Annoni, F. Ubiali, R. Longhi, R. | |
| | Mantegazza, F. Cornelio and C. Antozzi | |
| | in Ann. N. Y. Acad. Sci., 998 | |
| | pp. 395-8 (2003)) (EMG) | |
| m. | Acetylcholine receptor 259-271 (EMG) | VAL ILE VAL GLU |
| | (see an article entitled “Peptide Analogs | LEU ILE PRO SER |
| | to Pathogenic Epitopes of the Human | THR SER SER ALA |
| | Acetylcholine Receptor Alpha | VAL |
| | Subunit as Potential Modulators | |
| | of Myasthenia Gravis” by E. Zisman, | |
| | Y. Katz-Levy, M. Dayanm, S. L. | |
| | Kirshner et al in Proc. Nat. Acad. Sci., | |
| | USA, 93 pp. 4492-4496 (1996)) | |
| n. | Acetylcholine receptor 129-145 (EMG) | GLU ILE ILE VAL |
| | (see an article entitled “A 17-Mer Self- | THR HIS PHE PRO |
| | Peptide of Acetylcholine Receptor Binds | PHE ASP GLU GLN |
| | to B Cell MHC Class II. Activates Helper | ASN CYS SER MET |
| | T Cells, Stimulates Autoantibody | LYS |
| | Production and electrophysiologic | |
| | signs of myasthenia gravis” by H. | |
| | Yoshikawa, E. H. Lambert, | |
| | D. R. Walser-Kuntz, Y. Yasukawa, D. J. | |
| | Mc Cormick and V. A. Lennon in | |
| | J. Immunol., 159 pp. 1570-1577 (1997)) | |
| o. | Acetylcholine receptor 67-75 | TRP ASN PRO ASP |
| | (see an article entitled “The Main Region | ASP TYR GLY GLY |
| | of the Nicotinic Acetylcholine Receptor” | VAL LYS |
| | by M. Bellone, F. Tang, R. Milius and | |
| | B. M. Conti-Troncoi in J. Immunol., | |
| | 143 pp. 3568-3579 (1989)) | |
| p. | Sm B/B′ protein (ELSE) (see an article | PRO PRO PRO GLY |
| | entitled “Side-Chain Specificities and | MET ARG PRO PRO |
| | Molecular Modeling of Peptide | |
| | Determinants for Two Anti-Sm B/B′ | |
| | Autoantibodies” by J. A. James, | |
| | M. T. McClain, G. Koelsch, D. G. | |
| | Williams, and J. B. Harley in J. | |
| | Autoimmunity, 12 pp. 43-49 (1999)) | |

where: underlined amino acids make the most important contributions to autoimmuneactivity; the table does not list all the peptides which have been attributed to autoimmune disease induction, however, this is a representative group; underlined portions of some of the sequences refer to amino acids which are critical for activity; and approximately 25-30 different encephalitogenic regions have been described involving four (4) central nervous system proteins—the ones listed are the most carefully studied regions.

What is claimed is:

1. A method of treating Multiple Sclerosis, Rheumatoid Arthritis, Anorexia Nervosa or Autistic Spectrum Disorder comprises:
administering an effective amount of a medicament comprised of subtilisin to a mammal suffering from Multiple Sclerosis, Rheumatoid Arthritis, Anorexia Nervosa or Autistic Spectrum Disorder.

2. A method of treating Myasthenia Gravis, Uveoretinitis, Ulcerative Colitis, Celiac Disease, Anorexia Nervosa or Autistic Spectrum Disorder comprises:
administering an effective amount of a medicament comprised of oligopeptidase F (PepF) to a mammal suffering from Myasthenia Gravis, Uveoretinitis, Ulcerative Colitis, Celiac Disease, Anorexia Nervosa or Autistic Spectrum Disorder.

3. A method of treating Myasthenia Gravis, Lupus Erythematosus, Celiac Disease, Rheumatoid Arthritis or Autistic Spectrum Disorder comprises:
administering an effective amount of a medicament comprised of endopeptidase O2 (PepO2) to a mammal suffering from Myasthenia Gravis, Lupus Erythematosus, Celiac Disease, Rheumatoid Arthritis or Autistic Spectrum Disorder.

4. A method of treating Uveoretinitis, Ulcerative Colitis, Celiac Disease or Autistic Spectrum Disorder comprises:
administering an effective amount of a medicament comprised of endopeptidase O (PepO) to a mammal suffering from Uveoretinitis, Ulcerative Colitis, Celiac Disease or Autistic Spectrum Disorder.

5. A method of treating Celiac Disease or Autistic Spectrum Disorder comprises:
administering an effective amount of a medicament comprised of Oenicoccus oeni to a mammal suffering from Celiac Disease or Autistic Spectrum Disorder.

6. A method of treating Lupus Erythematosus or Rheumatoid Arthritis comprises:
administering an effective amount of a medicament comprised of endopeptidase O from Bifidobacterium animalis subsp lactis to a mammal suffering from Lupus Erythematosus or Rheumatoid Arthritis.

7. The method of claim 5 wherein the medicament further comprises endopeptidase O (PepO).

8. The method of claim 5 wherein the medicament further comprises endopeptidase O2 (PepO2).

9. The method of claim 5 wherein the medicament further comprises endopeptidase F (PepF).
